(19) United States
(12) Patent Application Publication
(10) Pub. No.: US 2024/0316164 A1
(43) Pub. Date: Sep. 26, 2024

(54) ENHANCED EXPRESSION OF AN AROMATIC AMINO ACID DECARBOXYLASE (AADC) IN THE SUBSTANTIA NIGRA FOR THE TREATMENT OF PARKINSON'S DISEASE

(71) Applicants: Northwestern University, Evanston, IL (US); Comell University, Ithaca, NY (US)

(72) Inventors: Dalton James SURMEIER, JR., Evanston, IL (US); Patricia GONZALEZ RODRIGUEZ, Evanston, IL (US); Michael J. KAPLITT, Ithaca, NY (US)

(21) Appl. No.: 18/575,597

(22) PCT Filed: Jul. 1, 2022
(86) PCT No.: PCT/US2022/073339

§ 371 Date: Dec. 29, 2023

Related U.S. Application Data
(60) Provisional application No. 63/217,557, filed on Jul. 1, 2021.

(51) Int. Cl. A61K 38/51; A61K 48/00; A61P 25/16; C12N 15/86

(52) U.S. Cl. CPC A61K 38/51; A61K 48/005; A61P 25/16; C12N 15/86; C12N 2750/14143; C12Y 401/01028

(57) ABSTRACT

Provided herein are methods and compositions for treating Parkinson's disease (PD), or alleviating symptoms thereof, by the enhancing expression of aromatic acid decarboxylase (AADC) in the substantia nigra. In particular, the beneficial effects of levodopa to subjects suffering from PD are extended by enhancing the expression of AADC in the substantia nigra of the subject.

### CROSS-REFERENCE TO RELATED APPLICATIONS

This application claims the benefit of U.S. Provisional Patent Application No. 63/217,557, filed on Jul. 1, 2021, which is incorporated by reference herein.

### SEQUENCE LISTING

The text of the computer readable sequence listing filed herewith, titled “39618-601_SEQUENCE_LISTING”, created Jun. 22, 2022, having a file size of 5,265 bytes, is hereby incorporated by reference in its entirety.

### FIELD

Provided herein are methods and compositions for treating Parkinson's disease (PD), or alleviating symptoms thereof, by the enhancing expression of aromatic acid decarboxylase (AADC) in the substantia nigra. In particular, the beneficial effects of levodopa to subjects suffering from PD are extended by enhancing the expression of AADC in the substantia nigra of the subject.

### BACKGROUND

In late-stage Parkinson's disease, patients become unresponsive to levodopa therapy. This results in a deterioration in the quality of life. The lack of responsiveness is attributable to the degeneration of dopaminergic neurons that express aromatic acid decarboxylase (AADC) that enzymatically converts levodopa to dopamine in the brain. There is an ongoing clinical trial that attempts to use viral gene delivery approaches to elevate the expression of AADC in the striatum to correct this defect.

### SUMMARY

Provided herein are methods and compositions for treating Parkinson's disease (PD), or alleviating symptoms thereof, by the enhancing expression of aromatic acid decarboxylase (AADC) in the substantia nigra. In particular, the beneficial effects of levodopa to subjects suffering from PD are extended by enhancing the expression of AADC in the substantia nigra of the subject.

In some embodiments, provided herein are methods of treating Parkinson's disease (PD) comprising increasing expression of an aromatic amino acid decarboxy lase (AADC) polypeptide in the substantia nigra in a subject suffering from PD. In some embodiments, the AADC polypeptide is capable of catalyzing the conversion of L-DOPA to dopamine. In some embodiments, the AADC polypeptide comprises at least 70% (e.g., 70%, 75%, 80%, 85%, 90%, 95%, 100%, or ranges therebetween) sequence identity with SEQ ID NO: 1. In some embodiments, increasing expression of the AADC polypeptide in the substantia nigra comprises administering an agent to the subject that produces localized increased expression of AADC. In some embodiments, the agent comprises a nucleic acid encoding the AADC polypeptide and administration results in expression of the AADC polypeptide from the nucleic acid. In some embodiments, the nucleic acid comprises 70% sequence identity with SEQ ID NO: 2. In some embodiments, the agent comprises a vector containing the nucleic acid encoding AADC. In some embodiments, the nucleic acid is DNA or RNA. In some embodiments, the vector is a viral vector. In some embodiments, the vector is a non-viral vector. In some embodiments, the vector is selected from a lipid nanoparticle, a plasmid, a transposon, an adeno-associated virus (AAV) vector, an adenovirus, a retrovirus, an integrating lentiviral vector (LVV), and a non-integrating LVV. In some embodiments, the agent is administered by injection into the substantia nigra. In some embodiments, injection consists of a single injection into the substantia nigra. In some embodiments, another injection of the agent is not performed for at least 1 week (e.g., 7 days 10 days, 14 days, 21 days, 28 days, etc., or ranges therebetween) prior to or after the single injection. In some embodiments, another injection of the agent is not performed for at least 30 days (e.g., 30 days, 40 days, 50 days, 60 days, 75 days, 100 days, or more, or ranges therebetween) prior to or after the single injection. In some embodiments, methods further comprise administering levodopa to the subject. In some embodiments, methods further comprise administering carbidopa to the subject.

In some embodiments, provided herein is the use of a nucleic acid or vector encoding AADC in the manufacture of a medicament for treatment of PD by administration to the substantia nigra. In some embodiments, provided herein is the use of a nucleic acid or vector encoding AADC as a medicament for treatment of PD by administration to the substantia nigra.

### BRIEF DESCRIPTION OF THE DRAWINGS

FIG. 1. Loss of Ndufs2 function triggers mitochondrial and genomic adaptations in SN DA Neurons. a, Schematic of a coronal section of the midbrain highlighting the sampled region of SNc (orange). b, Meta-analysis of RNAseq data obtained by Cre-dependent RiboTag mRNA precipitation from P40 wildtype mice. The heat map shows enrichment of dopaminergic-specific markers (N=5). c, Effective Ndufs2 knock-out was verified by qPCR on RiboTag harvested mRNA (wildtype N=5; cNdufs2^{-/-} N=5). d, TMRM labeled mitochondria of SN DA neuron in ex vivo brain slice. Scale bar: 10 μm. e, Representative TMRM fluorescence time series after the application of carboxyatractyloside, the adenine nucleotide transporter inhibitor (iANT). f, Box plots of TMRM after blocking ANT with carboxyatractyloside and depolarizing mitochondria with FCCP (wildtype, P40 n=8 (1 per section), 6 mice; cNdufs2^{-/-}, P20 n=4 (1 per section), 3 mice; P40 n=5 (1 per section), 4 mice)). g, Cartoon representing the activity of the ETC and ANT following Ndufs2 deletion. h, Electron micrographs of SN DA neurons. The nucleus and the mitochondria are highlighted in green and red, respectively. Scale bar: 1 μm. i, Box plots showing no differences in mitochondrial density (wildtype n=21; cNdufs2^{-/-} n=21). j, Box plots showing mitochondrial morphology in wildtype and MCI-Park SN neurons (wildtype n=21; cNdufs2^{-/-} n=21). k-l, representative images showing normal and abnormal mitochondria. The percentage of abnormal mitochondria was calculated as the ration between the area occupied by abnormal mitochondria over the total area occupied by mitochondria for each cell. Scale bar: 0.2 μm. Heat maps of RNASeq expression show down-regulation of OXPHOS (m) and up-regulation of glycolysis (n) in cNdufs2^{-/-} mice (wildtype N=5; cNdufs2^{-/-} N=6 mice). o, Representative image of SN DA neurons expressing Perceval-HR and stained for TH in ex vivo brain slice. Scale bar: 15 μm. p, Representative time-lapse measurements of Perceval-HR fluorescence ratio. Oligomycin and 2-deoxyglucose were applied to determine OXPHOS and glycolytic contribution to ATP/ADP ratio. q, Box plots show OXPHOS index (OXPHOS/(OXPHOS+glycolysis)) (wildtype n=6; cNdufs2^{-/-} n=10). Wildtype (grey); cNdufs2^{-/-} (black). *P<0.05; **P<0.01; ***P<0.001; ****P<0.0001 Mann-Whitney test; (c), (i), (j), (q); Kruskal-Wallis test (f). Data are presented as box-and-whisker plots depicting median, quartiles, and range.

FIG. 2. Loss of Ndufs2 induces an early axonal dysfunction. a, Heat map of RNASeq shows down-regulation of dopamine release and synaptic function in cNdufs2^{-/-} mice (wildtype N=5; cNdufs2^{-/-} N=6). b-c, Representative images showing a significant reduction in TH staining in dorsal striatum but not in SNc in P30 cNdufs2^{-/-} mice. Scale bars: 1 mm (top) and 200 μm (bottom). d, Quantification of TH expression in dorsal striatum (wildtype N=5; cNdufs2^{-/-} N=5). e, Quantification of TH expression in SNc and VTA (wildtype N=5; cNdufs2^{-/-} N=5). Striatal (f) and dendritic (g) dopamine release measured with fast-scan cyclic voltammetry (f) and dLight1.3b measurements (g). Solid lines represent median trace, shaded area is 95% CI. Scale bars: (f) 0.5 μM dopamine, 1 s. (g) 5%ΔΕ/F0, 10 s. h-i, Quantification of dopamine release at P30 in dorsal striatum (h, wildtype N=4; cNdufs2^{-/-} N=4) and SN (i, wildtype N=6; cNdufs2^{-/-} N=9). j, Cell-attached recordings from identified wildtype and cNdufs2^{-/-} SN DA neurons. Scale bars: 10 pA, 1 s. k, Cumulative probability plot of autonomous discharge rates (wildtype n=21, cNdufs2^{-/-} n=21). 1, Left, representative reconstruction of Fura-2 filled SN neuron. Scale bar: 20 μm. Right, whole-cell recording and Fura-2 Ca2+-imaging from wildtype and cNdufs2^{-/-} SN DA neurons at P30. Scale bars: 20 mV, 1 s. m, Cumulative probability plot of peak [Ca2+] at proximal dendrite (wildtype n=8; cNdufs2^{-/-} n=6). n, Whole-cell somatic recordings showing the response to glutamate uncaging. Scale bars: 20 mV. Is. Representative SN DA neuron filled with Alexa Flour 594 showing the location for uncaging in blue. Scale bar: 20 μm. o, Spikes/burst-peak spiking rate plot showing the difference in response to uncaged glutamate between wildtype (n=5) and cNdufs2^{-/-} (n=5). Wildtype (grey); cNdufs2^{-/-} (black). *P<0.05; **P<0.01; Mann-Whitney test; (d), (e), (h), (i), (k), (m) and (o). Data are presented as box-and-whisker plots depicting median, quartiles, and range.

FIG. 3. Progressive loss of somatodendritic phenotype, but not neuronal death. a-b, Representative images showing TH immunostaining in the striatum in wildtype (a) and cNdufs2^{-/-} (b) at P60. Scale bar: 1 mm. c, Quantification of TH expression in dorsal striatum (wildtype N=5; cNdufs2^{-/-} N=5). d-e, Representative images showing a significant reduction for TH staining in SNc in cNdufs2^{-/-} mice at P60. Scale bar: 200 μm (top). Higher magnification images of SNc showing DA neurons in wildtype and cNdufs2^{-/-}. Scale bar: 15 μm. f, Quantification of TH expression in SNc (wildtype N=5; cNdufs2^{-/-} N=5). Striatal (g) and dendritic (h) dopamine release measured with fast-scan cyclic voltammetry (g) and dLight1.3b measurements (h). Solid lines represent median trace, shaded area is 95% CI. Scale bars: (g) 0.5 μM of dopamine. 1 s; (h) 5% ΔF/F0, 10 s. (i-j) Quantification of dopamine release at P60 in dorsal striatum (i, wildtype n=9); cNdufs2^{-/-} n=4) and SN (j, wildtype n=5; cNdufs2^{-/-} n=9). k, Diagram of Fluoro-Gold (FG) injection for analysis of retrograde transport. l-m. Representative images showing phenotypic down-regulation but not cell loss in brain sections obtained from wildtype (N=4) (1) and cNdufs2^{-/-} (N=4) (m) mice at P60. TH-IR (red) and FG (green) in SN of wildtype and cNdufs2^{-/-} sections. Scale bars: 20 μm. n, Cell-attached recordings from identified wildtype and cNdufs2^{-/-} SN DA neurons at P60. Scale bar: 10 pA. 1s. o, Cumulative probability plot of autonomous discharge rates (wildtype n=20; cNdufs2^{-/-} n=13 cells). p, Whole-cell somatic recordings from a cNdufs2-SN DA neurons at P60 showing the response to glutamate uncaging. Representative SN DA neuron filled with Alexa Flour 594 is showing the location for uncaging in blue. Scale 20 mV. 2s. 20 μm. q, Spikes/burst-peak spiking rate plot showing the difference in response to uncaged glutamate between wildtype (n=4) and cNdufs2^{-/-} (n=5). Wildtype (grey); cNdufs2^{-/-} (black). *P<0.05; **P<0.01; Mann-Whitney test; (c), (f), (i), (j), (o) and (q). Data are presented as box-and-whisker plots depicting median, quartiles, and range.

FIG. 4. cNdufs2^{-/-} mice manifest a progressive, levodopa-responsive Parkinsonism. a, Schematic diagram of the experimental protocol for Y maze test. The scoring of 2 and 3 was counted as incorrect. b, Box plots indicate the percentage of correct choices for wildtype (P30 N=8; P60 N=6) cNdufs2^{-/-} (P30 N=8; P60 N=6), and cNdufs2^{-/-} plus 6 mg/kg levodopa (P30 N=8; P60 N=6). c, Striatal dopamine release measured with fast-scan cyclic voltammetry at P30 and P60. Local electrical stimulation (350 μA, 2 ms) was used to evoke release. Levodopa pretreatment increased DA release to measurable levels in P30 but not P60 mice (P30, vehicle N=5, levodopa N=9); P60, vehicle N=4, levodopa N=6). d. Adhesive removal test from P15 to P120, cNdufs2^{-/-} mice at P15 showed no difference in the time to remove the adhesive compared to wildtype. By P30, cNdufs2^{-/-} mice took longer (N=10-13 per group). e, Open-field traces in wildtype and cNdufs2^{-/-} mice at P30 and P100. f, Number of pauses during open field test in wildtype and cNdufs2^{-/-} mice. By P40, cNdufs2^{-/-} showed increases in number of pauses. Levodopa (3 mg/kg) rescued this deficit (N=10-12 per group). g, Total distance traveled in 5 min. N=12-27per group. h-m, Gait analysis procedure and measurement schematics. h, Pictures of wildtype and cNdufs2^{-/-} mice footprints with representations of measurements of RF (Right Fore). RH (Right Hind). LF (Left Fore) and LH (Left Hind) in yellow. i, Box plots indicate body length measurements made along the long axis of the mouse from nose to base of tail (N=7 per group). j, Box plots show hind limb stance width for wildtype, cNdufs2^{-/-} and cNdufs2^{-/-}+6 mg/kg levodopa at P100. Levodopa rescued this phenotype (N=7 per group). k, Representative graph of right hind paw area in contact with the treadmill (17 cm/see) surface over time. l. Box plots represent stride length in right hind paw at P100 (N=7 per group). m, Boxplots indicate swing duration in right hind paw. This deficit was not rescued by levodopa (N=6-7 per group). n, Schematic diagram showing behavioral differences between young and older cNdufs2^{-/-} mouse. Wildtype [gray], cNdufs2^{-/-} [black], cNdufs2^{-/-} plus 6 mg/kg levodopa [red]. *P<0.05; **P<0.01, (*wildtype vs cNdufs2^{-/-}); #P<0.01 (#cNdufs2^{-/-} vs cNdufs2^{-/-}+6 mg/kg levodopa). Kruskal-Wallis test: (b), (d), (f) and (g). ***P<0.001. 2-way ANOVA followed by Tukey's post hoc test (c). *P<0.05; **P<0.01; 1-way ANOVA followed by Tukey's post hoc test (i), (j), (l) and (m). Data are presented as box-and-whisker plots depicting median, quartiles, and range (b), (c), (i), (j), (l) and (m) or median and range (shaded area); (d), (f) and (g).

FIG. 5. Boosting mesencephalic dopamine levels reversed motor deficits in cNdufs2^{-/-} mouse. a, (Top) Schematic diagram showing the conversion of levodopa to dopamine by aromatic amino acid decarboxylase (AADC). (Bottom) Experimental timeline for the AADC experiments. b, Schematic diagram of the striatal injection strategy. AAV2-GFP-AADC or AAV2-GFP was bilaterally injected into the striatum of cNdufs2^{-/-} mice at P60. c, Confocal image of a coronal slice showing robust expression of GFP in the striatum of a cNdufs2^{-/-} mouse injected with AAV2-GFP-AADC. Scale bar: 200 μm. d, Magnified view of the dorsal striatum showing strong expression of AADC-GFP in medium spiny neurons in cNdufs2^{-/-} mice. Scale bar: 20 μm. e, Schematic diagram of the SN injection strategy. AAV2-GFP-AADC or AAV2-GFP was bilaterally injected into the SN of cNdufs2^{-/-} mice at P60. f, Confocal image of a coronal slice showing expression of AADC-GFP in the SN of a cNdufs2^{-/-} mouse at P100. Scale bars: 500 μm. g-h, Magnified views of SN showing GFP+ neurons in cNdufs2^{-/-}. Scale bar: 100 μm (g), 20 μm (h). i, Quantification of dopamine in striatal tissue by HPLC-LC/MS from wildtype (N=4), cNdufs2^{-/-}+low levodopa (1.5 mg/kg) with (N=4) or without AADC (N=4) and cNdufs2^{-/-}+high levodopa (12 mg/kg) (N=4) at P100. j, Schematic diagram of the AAV-AADC injection into the SN and AAV-dLight injection into the striatum. cNdufs2^{-/-} mice were injected at P60 and experiments were performed at P100. k, dLight fluorescence (raw in thin lines and average in thick lines) in response to a single electrical stimulus (350 μA, 2 ms). Upper: 5 slices from a wildtype mouse; middle; 4 slices from a cNdufs2^{-/-} mouse injected with AAV-AADC in the SN; lower: 3 slices from a Ndufs2^{-/-} mouse injected with AAV-GFP in the SN. 1. Summary of dLight responses (wildtype=16 slices from 4 mice, cNdufs2^{-/-} −AADC=12 slices from 4 mice, cNdufs2^{-/-}+AADC=9) slices from 3 mice). *P<0.05. Mann-Whitney test. m-n, Representative traces of locomotor activity in the open field test of cNdufs2^{-/-} mice at P100 before and after levodopa treatment (1.5 mg/kg). o, Box plots showing the total distance traveled during 5 minutes for cNdufs2^{-/-} mice infected with AAV2-GFP (black) or AAV2-GFP-AADC (red). A single AAV injection was performed for SN. For the striatum, two injection strategies were tested: a single AAV injection (striatal) or 3 AAV injections to cover the whole striatum (X3 striatal). SN or striatal AADC expression improve levodopa response (N=6-7 per group). p, A schematic showing our hypothesis regarding the cascade of events in the prediagnostic phase (prodromal stage) and clinical phase of Parkinson's disease. Wildtype (grey); cNdufs2^{-/-} (black); cNdufs2^{-/-}+AADC (red); cNdufs2^{-/-}+high levodopa (blue). *P<0.05; **P<0.01; ***P<0.001. (i). (o). Two-way ANOVA followed by Tukey's post hoc test. (1) ****, p<0.0001. Kruskal-Wallis with Dunn's correction for multiple comparisons. Data are presented as box-and-whisker plots depicting median, quartiles, and range.

FIG. 6. Generation of cNdufs2^{-/-} mice. (a-b). Ndufs2 was ablated specifically in dopaminergic neurons by selective breeding of mice expressing Cre under the control of the dopamine transporter (DAT) promoter with mice containing a floxed allele of the Ndufs2 gene.

FIG. 7. Loss of Ndufs2 decreases OXPHOS in ventral tegmental area dopaminergic neurons and dopaminergic terminals. (a)Top, schematic drawing of a coronal section of the midbrain, positioned 3.52 mm posterior to bregma. Bottom, magnified view focusing on the ventral part and highlighting the sampled region of ventral tegmental area (VTA) (red). b, Box plots indicate that OXPHOS index (OXPHOS/(OXPHOS+glycolysis)) is lower in Ndufs2 deficient neurons (wildtype, n=5; cNdufs2^{-/-} n=7). c, Confocal image of dopaminergic terminals in wildtype mice expressing Perceval-HR in ex vivo brain slice at P40. Scale bar: 20 μm. d, Box plots show decrease in the OXPHOS index (OXPHOS/(OXPHOS+glycolysis)) in dopaminergic terminals of cNdufs2″ mice (wildtype, n=4; cNdufs2^{-/-}, n=5). *P<0.05. Mann-Whitney test. Data are presented as box-and-whisker plots depicting median, quartiles, and range.

FIG. 8. Bar graph of enrichment analysis in down-regulated genes in MCI-Park mice. Bar graph for viewing top 20 enrichment clusters, one per cluster, using a discrete color scale to represent statistical significance (Metascape). Wildtype. N=5; cNdufs2^{-/-}. N=6.

FIG. 9. Bar graph of enrichment analysis in up-regulated genes in MCI-Park mice. Bar graph for viewing top 20 enrichment clusters, one per cluster, using a discrete color scale to represent statistical significance (Metascape). Wildtype. N=5; cNdufs2^{-/-}. N=6.

FIG. 10. Enrichment network visualization in down-regulated genes in MCI-Park mice. Network of enriched terms colored by cluster identity, where nodes that share the same cluster identity are typically close to each other (Metascape). Wildtype. N=5; cNdufs2^{-/-} N=6.

FIG. 11. Enrichment network visualization in up-regulated genes in MCI-Park mice. Network of enriched terms colored by cluster identity, where nodes that share the same cluster identity are typically close to each other (Metascape). Wildtype. N=5; cNdufs2^{-/-} N=6.

FIG. 12. TH expression in SNc and VTA dopaminergic neurons in wildtype and cNdufs2^{-/-} mice. (a) Quantification of TH expression in SNc dopaminergic neurons at P30 and P60 (wildtype N=5; cNdufs2^{-/-} N=5). b, Quantification of TH expression in VTA dopaminergic neurons at P30 and P60 (wildtype N=5; cNdufs2^{-/-} N=4). *P<0.05; **P<0.01. Mann-Whitney test (a) and (b). Data are presented as box-and-whisker plots depicting median, quartiles, and range

FIG. 13. Reduced striatal dopamine transporter in cNdufs2^{-/-} mice. a-b, Representative images showing a significant reduction in dorsal striatum, but not in SNc, for dopamine transport (DAT) in P30 cNdufs2^{-/-} mice. (wildtype. N=3; cNdufs2^{-/-} N=3). Scale bars: 1 mm and 200 μm.

FIG. 14. Expression of dLight1.3b in SN DA neurons and dorsal striatum. Mice were injected into SN with AAV-TH-ERtdTomato and AAV-syn-dLight1.3b. Representative images from SN in P30 wildtype (a) and cNdufs2^{-/-} (b) mice are shown. Scale=5 μm. c-d, As ER is difficult to visualize in axons by light microscopy, mice were injected with AAV-TH-FusionRed and AAV-syn-dLight1.3b into the SN. Representative images from dorsal striatum in wildtype (c) and cNdufs2^{-/-} (d) mice are shown. Scale=5 μm. Note that at P30, the TH promoter (not TH expression) was effectively driving the expression of both dLight and fluorescent reporters in dopaminergic neurons, despite the down-regulation in TH expression in the dorsolateral striatum (shown in FIG. 2c).

FIG. 15. Dopamine release is reduced in cNdufs2^{-/-} mice. (a-c) Dopamine release was measured by fast-scan cyclic voltammetry (FSCV) in wildtype and cNdufs2^{-/-} mice at P20. Representative colorplots (a) and traces (b) show dramatic reduction in evoked (1p. 350) nA, 2 ms) release in dorsal striatum of cNdufs2″ mice compared to wildtype. Scale: vertical=100 nM dopamine, horizontal=1 sec. c, Summary data demonstrate dopamine release is significantly decreased by P20 (wildtype N=12, cNdufs2^{-/-} N=4) ****P<0.0001. Mann-Whitney test. (d-i) Striatal dopamine release measured with dLight1.3b at P30 (d, f, h) and P60 (e, g, i). Traces are ΔF/F0 over time. Solid lines represent median trace, shaded area is 95% CI. Scale bars: (d, e), vertical=200% ΔF/F0, horizontal=500 ms. (f-l). Quantification of dopamine release at P30 (f, h) and P60 (g, i) in dorsal striatum; striatal dLight1.3b responses were analyzed either by defining 16-pixel-wide line profiles, which provided high temporal resolution measurements of selected regions (f-g), or by averaging the entire field of view, with lower temporal resolution but broader sampling area (h, i). Both approaches detected no dopamine release in cNdufs2^{-/-} mice striatum either at P30 or P60 (f, wildtype N=5; cNdufs2^{-/-} N=5; g, wildtype N=6; cNdufs2^{-/-} N=6; h, wildtype N=4; cNdufs2^{-/-} N=5; i, wildtype N=9; cNdufs2^{-/-} N=4). *P<0.05; Mann-Whitney test: (f), (g), (h) and (i). Data are presented as box-and-whisker plots depicting median, quartiles, and range.

FIG. 16. Striatal dopamine drops in parallel with dopamine release in cNdufs2^{-/-} mice. Quantification of neurotransmitters in striatal tissue by HPLC-LC/MS from wildtype and cNdufs2^{-/-} at P30 and P120. Dopamine (a), DOPAC (b), serotonin (c), and acetylcholine (d) separated from wildtype and cNdufs2^{-/-} striatum tissue lysate (wildtype N=4 per group, cNdufs2^{-/-} N=4 per group). Note that elevation of striatal serotonin was detected at P120. This is a common feature of rodent PD models60. Wildtype (grey); cNdufs2″ (black). *P<0.05; ***P<0.001; Mann-Whitney test. Data are presented as box-and-whisker plots depicting median, quartiles, and range.

FIG. 17. Physiology remodeling in cNdufs2^{-/-} mice. (a) Heat maps illustrating the remodeling of ions channels in cNdufs2^{-/-} mice; repeated samples are grouped horizontally (wildtype. N=5; cNdufs2^{-/-} N=6). b, q-PCR of RiboTag harvested mRNA showing a drop in Cav1.3 mRNA in cNdufs2^{-/-} SN dopaminergic neurons (wildtype N=4; cNdufs2^{-/-} N=4) c, q-PCR of RiboTag harvested mRNA showing a drop in HCN2 mRNA in cNdufs2^{-/-} SN dopaminergic neurons (wildtype N=4; cNdufs2^{-/-} N=4). d, Whole-cell somatic recording showing hyperpolarization-activated, cyclic nucleotide-gated (HCN) currents from a wildtype and cNdufs2^{-/-} neuron at P30. Scale bars: 100 pA, 200 ms. e, Cumulative probability plot of peak current from wildtype and cNdufs2^{-/-} SN dopaminergic neurons (wildtype, n=12; cNdufs2^{-/-}. n=10). Scale bars: 20 mV, 1s. f, Representative traces showing spike width in SN neurons from wildtype and cNduf2^{-/-} at P30. (g) Box plots indicate AP half width in wildtype and cNduf2^{-/-} at P30 (wildtype n=6; cNdufs2^{-/-} n=7). Data are presented as box-and-whisker plots depicting median, quartiles, and range

FIG. 18. Reduction of TH expression in VTA dopaminergic neurons in cNdufs2^{-/-} mice. (a) Representative images showing TH-IR in VTA and SN dopaminergic neurons in wildtype mouse at P60. Scale bar: 200 μm. b, Magnified VTA region showing dopaminergic neurons in wildtype at P60. Scale bar: 15 μm. c, Representative images showing TH-IR in VTA and SN dopaminergic neurons in cNdufs2^{-/-} mouse at P60. Scale bar: 200 μm. d, Magnified VTA region showing dopaminergic neurons in cNdufs2^{-/-} mouse at P60. Scale bar: 15 μm. e, Quantification of TH expression in VTA dopaminergic neurons (wildtype N=4; cNdufs2^{-/-} N=4). Data are presented as box-and-whisker plots depicting median, quartiles, and range

FIG. 19. Electron microscopy of SN dopaminergic neurons in cNdufs2^{-/-} mice. (a), (b), Electron micrographs of SN dopaminergic neurons at P60. The nucleus and the mitochondria are highlighted in green and red, respectively. Scale bar: 2 μm. c, Box plots showing no differences in mitochondrial density (wildtype n=14; cNdufs2^{-/-} n=23). d, Box plots showing abnormal morphology in MCI-Park mitochondria (wildtype n=14; cNdufs2^{-/-} n=23). (e) Representative images showing normal and abnormal mitochondria. The percentage of abnormal mitochondria was calculated as the ratio between the area occupied by abnormal mitochondria over the total area occupied by mitochondria for each cell. Scale bar: 500 nm. Data are presented as box-and-whisker plots depicting median, quartiles, and range.

FIG. 20. Identification of SN dopaminergic neurons in cNdufs2^{-/-} mice. (a) Schematic diagram of injection site. TH-Fusion Red reporter was bilaterally injected into the SN of cNdufs2^{-/-} mouse at P50. Experiments were done at P60 (+4 days). b, Representative image showing TH-Fusion Red expression in wildtype mouse at P60. Scale bar: 20 μm. c, Representative image showing TH-Fusion Red expression in cNdufs2^{-/-} mouse at P60. Scale bar: 20 μm (wildtype. N=5; cNdufs2^{-/-} N=5).

FIG. 21. Behavioral phenotypes in cNdufs2^{-/-} mice. (a) Rearing test performance. b. Number of rearings in a 3-minute period in wildtype, cNdufs2^{-/-} and cNdufs2^{-/-}+3 mg/kg levodopa mice at different ages (P20-P120). Number of rearings begin to be impaired at P40. Levodopa did not rescue this deficit (N=12-13 per group) c. Rearing time in wildtype. cNdufs2^{-/-} and cNdufs2^{-/-}+3 mg/kg levodopa mice at different ages (P20-P120). At P60, cNdufs2^{-/-} mice show difficulty transitioning between rearing and landing, spending much more time ‘stuck’ in an elevated posture. Levodopa did not rescue this deficit (N=12-13 per group). d, Open-field traces in wildtype and cNdufs2^{-/-} mice at P60 and P120 with and without levodopa (3 mg/Kg) treatment. e-g, Representatives traces showing the effect of levodopa treatment on the speed in wildtype and cNdufs2^{-/-} mice at P120. h-o, Gait analysis procedure and measurement schematics. h, Step sequence in right hind paw for wildtype, cNdufs2^{-/-} and cNdufs2^{-/-}+6 mg/kg levodopa at P30. P60 and P100. Levodopa did not rescue this phenotype (N=6-8/group/time point). i, Stance duration in right hind paw for wildtype, cNdufs2^{-/-} and cNdufs2^{-/-}+6 mg/kg levodopa at P30. P60 and P100. Levodopa rescued this phenotype at P100 but not at P60 (N=6-8/group/time point). j, Brake duration in right hind paw for wildtype, cNdufs2^{-/-} and cNdufs2^{-/-}+6 mg/kg levodopa at P30. P60 and P100. Levodopa did not rescue this phenotype (N=6-8/group/time point). k, Paw area in right hind paw for wildtype, cNdufs2^{-/-} and cNdufs2^{-/-}+6 mg/kg levodopa at P30. P60 and P100. Levodopa rescued this phenotype at P100 but not at P60 (N=6-8/group/time point). 1. Stride frequency in right hind paw for wildtype, cNdufs2^{-/-} and cNdufs2^{-/-}+6 mg/kg levodopa at P30. P60 and P100. Levodopa did not rescue this phenotype (N=6-8/group/time point). m, Propulsion time in right hind paw for wildtype, cNdufs2^{-/-} and cNdufs2^{-/-}+6 mg/kg levodopa at P30. P60 and P100. Levodopa did not rescue this phenotype (N=6-8/group/time point). n, Gait symmetry in right hind paw for wildtype and cNdufs2^{-/-} mice at P30, P60 and P100. Gait symmetry shows no differences between wildtype and cNdufs2^{-/-} mice (N=6-8/group/time point). o, Fore limb stance width (LF-RF) for wildtype and cNdufs2^{-/-} mice at P30. P60 and P100. Fore limb stance width (LF-RF) shows no differences between wildtype and cNdufs2^{-/-} mice (N=6-8/group/time point). Wildtype [gray], cNdufs2^{-/-} [black], cNdufs2^{-/-} plus levodopa [red]. *P<0.05; **P<0.01. (*wildtype vs cNdufs2^{-/-}); #P<0.01 (#cNdufs2^{-/-} vs cNdufs2^{-/-} +levodopa). Kruskal-Wallis test: (b), and (c). *P<0.05; **P<0.01; 1-way ANOVA followed by Tukey's post hoc test: (h-o). Data are presented as median and range (shaded area).

FIG. 22. Growth curves of wildtype and cNdufs2^{-/-} mice. Body weight was analyzed from P20 to P120. a, Body weight development of wildtype and cNdufs2^{-/-} mice in males. b, Body weight development of wildtype and cNdufs2^{-/-} mice in females. (male: N=10-25/group/time point; female: N=9-22/group/time point). *P<0.05; **P<0.01; 1-way ANOVA followed by Tukey's post hoc test (a) and (b). Data are presented as median and range (shaded area).

FIG. 23. Stereological estimation of dopaminergic neuron number in the SNc and VTA. (a) Box-plots summarizing stereological estimate of the numbers of NeuN-immunopositive neurons in the SNC (a) and VTA (b). In cNdufs2^{-/-} (N=5, age P120-P50) there was significant decrease in neuronal number compared to wildtype mice (N=5, age P120-P50). c, Representative images showing NeuN immunostaining in the midbrain in wildtype (top panel) and cNdufs2^{-/-} mouse (bottom panel) in P120-150 mice. Midbrain dopaminergic groups were outlined on the basis of TH immunolabeling, with reference to a coronal atlas of the mouse brain (Paxinos and Franklin, 2013). Scale bar: 100 μm. Wildtype (grey); cNdufs2^{-/-} (black); **P<0.01; Mann-Whitney test (a) and (b). Data are presented as box-and-whisker plots depicting median, quartiles, and range.

FIG. 24. Target-specific expression of AADC-GFP protein in cNdufs2^{-/-} mouse. (a) Schematic diagram of injection site. AAV2-GFP-AADC was bilaterally injected into the striatum of cNdufs2^{-/-} mouse at P60. Analysis was done at P100. b, Confocal image of coronal slice containing striatum from cNdufs2^{-/-} mouse injected with AAV2-GFP-AADC at P100. A robust expression of GFP in the striatum was observed in P100 cNdufs2^{-/-} mouse. Scale bar: 200 μm, c, Confocal image of coronal slice containing SN from cNdufs2^{-/-} mouse injected into the striatum with AAV2-GFP-AADC at P100. AADC-GFP expression was not observed in SN after striatal injection. Scale bar: 200 μm. d, Magnified view of SN showing no expression of AADC-GFP after striatal injection in cNdufs2^{-/-} at P100. Scale bar: 20 μm. e, Schematic diagram of injection site. AAV2-GFP-AADC was bilaterally injected into the SN of cNdufs2^{-/-} mouse at P60. f, Confocal image of coronal slice containing striatum from cNdufs2^{-/-} mouse injected into SN with AAV2-GFP-AADC at P100. No expression of AADC-GFP in the striatum after SN injection was observed at P100 cNdufs2^{-/-} mouse. Scale bar: 200 μm. g, Confocal image of coronal slice containing SN from cNdufs2^{-/-} mouse injected into the SN with AAV2-GFP-AADC at P100. A robust expression of GFP in the SN was observed at P100 cNdufs2^{-/-} mouse. Scale bar: 200 μm. h, Magnified view of SN showing expression of AADC-GFP after SN injection in cNdufs2^{-/-} mouse at P100. Scale bar: 20 μm.

FIG. 25. SN expression of AADC did not induce DA release in P100 cNdufs2^{-/-} mice even after perfusion with dopamine. (a) An example recording of dLight fluorescence response upon bath application of dopamine (100 μM) followed by washout in a cNdufs2^{-/-} mouse injected with AADC in the SN. b-c, Example recordings of dLight fluorescence responses (raw in thin lines and average in thick lines) upon single electrical stimuli (350 μA, 2 ms) after dopamine washout. (b), 4 slices from a cNdufs2^{-/-} mouse injected with AADC in the SN; (c), 3 slices from a Ndufs2^{-/-} mouse injected with GFP in the SN.

FIG. 26. Schematic showing the cascade of events following to Ndufs2 deletion in MCI-Park mice. Mitochondrial complex I dysfunction in dopaminergic neurons triggers a cascade of pathological events, which begins in axons and ends in the somatodendritic compartment; this sequence drives region-specific network dysfunction and staged behavioral deficits. Data are shown as % relative to wildtype from P20 to P120. DA (dopamine), ETC (electron transport chain), DLS (dorsolateral striatum).

### DETAILED DESCRIPTION

Provided herein are methods and compositions for treating Parkinson's disease (PD), or alleviating symptoms thereof, by the enhancing expression of aromatic acid decarboxylase (AADC) in the substantia nigra (SN). In particular, the beneficial effects of levodopa to subjects suffering from PD are extended by enhancing the expression of AADC in the substantia nigra of the subject.

The most potent medication for Parkinson's disease (PD) is levodopa (L-Dopa). In late-stage Parkinson's disease, patients become unresponsive to levodopa therapy. This results in a deterioration in the quality of life. The lack of responsiveness is attributable to the degeneration of dopaminergic neurons that express aromatic acid decarboxylase (AADC) that enzymatically converts levodopa to dopamine in the brain. Enhancing expression of AADC in the brain prolongs the responsiveness of patients to levodopa treatment. Provided herein are compositions and methods for targeting the substantia nigra, rather than the striatum, with the AADC gene therapy. The substantia nigra is smaller and accessible, allowing a single viral injection to effectively cover the region in contrast to the striatum, which requires multiple injections.

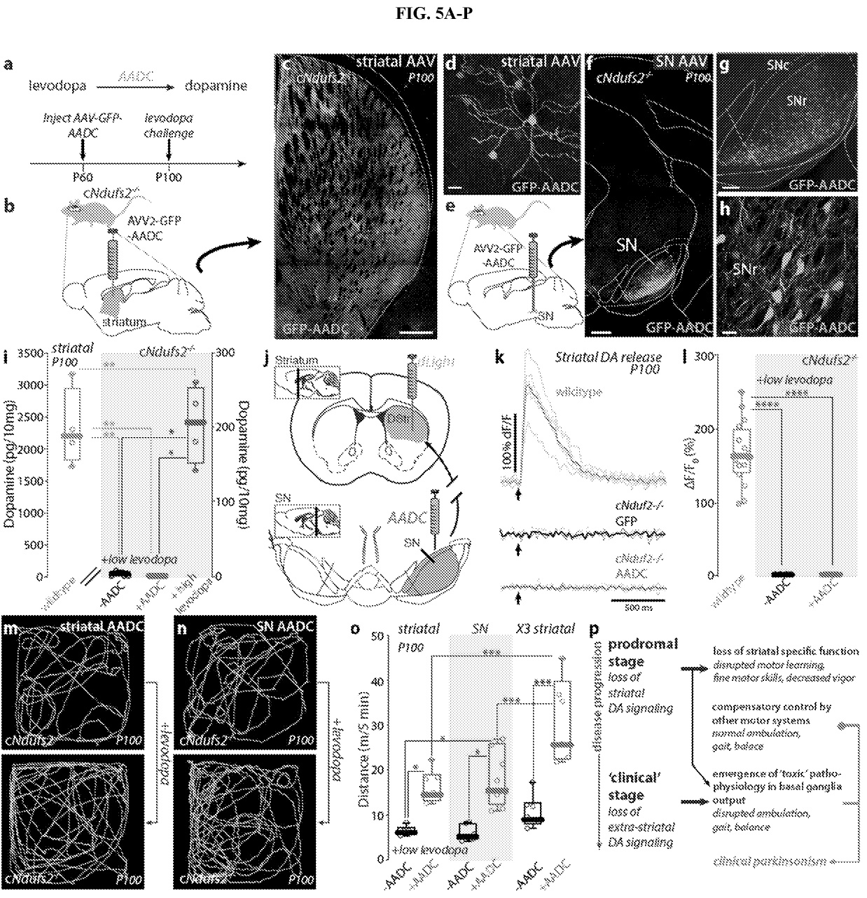

Experiments were conducted during development of embodiments herein to using the MCI-Park mouse. Over several months, this mouse faithfully recapitulates the staging of basal ganglia pathology seen in PD patients. In the initial stages of pathology, nigrostriatal axons lose the ability to release dopamine in the striatum. With this deficit, mice manifest learning deficits, and the ability to perform fine motor task, but the ability to ambulate in the open field is intact. Weeks later, pathology manifests itself in the somatodendritic regions, resulting in a loss of SN dopamine release. It is at this point that ambulation becomes impaired, resembling that seen in PD patients at diagnosis. Based on these observations, it is contemplated that restoration of SN dopamine levels will be sufficient to alleviate open-field, ambulatory deficits that plague PD patients. Alternatively, striatal dopamine depletion is necessary but not sufficient for ambulatory deficits-contrary to prevailing models and treatment plans. Experiments were conducted during development of embodiments herein to administer an adenoassociated virus (AAV) carrying an AADC expression construct under the control of a generic promoter, by injecting the vector into either the striatum or SN of a late-stage MCI-Park mice (P60) (one that was parkinsonian). These stereotaxic injections were region-specific and limited in volume, restricting AADC expression in a manner mimicking the approach used in humans (FIG. 5). After allowing time for AADC expression (˜P100), mice injected with the AADC or a reporter construct were given a low dose of levodopa (that had no effect in control mice) and tested in the open field. Striatal expression of AADC significantly improved open-field activity (FIG. 5). Mesencephalic AADC expression was as effective as the striatal injection in alleviating the ambulatory deficit in late-stage MCI-Park mice. As it does not cross the blood-brain barrier, dopamine generated from levodopa in the mesencephalon or striatum stays in that region. The experiments conducted during development of embodiments herein indicate that rather than targeting the striatum, which is large and hard to cover with an AAV, the much smaller and more tractable SN is a better site for the AADC injection. The SN is smaller and accessible, allowing a single viral injection to effectively cover the region, limiting surgery time and potential brain damage resulting from multiple injections.

There exists a long-felt and unmet need for compositions and methods suited for, without limitation, treating PD, alleviating symptoms of PD, and/or prolonging the therapeutic effects of levodopa on patients suffering from PD. In some embodiments, the present disclosure provides such compositions and methods.

Aromatic L-amino acid decarboxylase (AADC or AAAD), also known as DOPA decarboxylase (DDC), tryptophan decarboxylase, and 5-hydroxytryptophan decarboxylase, is a lyase enzyme. AADC catalyzes several different decarboxylation reactions, including L-Dopa to dopamine.

The present disclosure provides polynucleotides (e.g., DNA, RNA, etc.) encoding AADC. The present disclosure provides vectors comprising such polynucleotides and, in some embodiments, one or more additional nucleic acids encoding other proteins. The disclosed polynucleotides are useful, for example, in enhancing expression of AADC in cells, tissues, etc. to which they are administered. In some embodiments, the polynucleotides are provided as vectors for administration (e.g., injection) and expression of the encoded AADC. In some embodiments, the vectors comprise other functional elements useful for the stabilization, localization, cellular uptake, and/or expression (e.g., promoter) of AADC.

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a vector” is a reference to one or more vectors and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition.

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “co-administration” and “co-administering” refer to the administration of at least two agent(s) (e.g., a vector encoding AADC and levodopa) or therapies to a subject. In some embodiments, the co-administration of two or more agents or therapies is concurrent (e.g., in a single formulation/composition or in separate formulations/compositions). In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

“Treatment,” “treating,” and “treat” are defined as acting upon a disease, disorder, or condition with an agent to reduce or ameliorate harmful or any other undesired effects of the disease, disorder, condition and/or their symptoms.

As used herein, the term “pharmaceutical composition” refers to the combination of an active agent with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintigrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. (1975), incorporated herein by reference in its entirety.

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

As used herein, the term “instructions for administering said compound to a subject,” and grammatical equivalents thereof, includes instructions for using the compositions contained in a kit for the treatment of conditions (e.g., providing dosing, route of administration, decision trees for treating physicians for correlating patient-specific characteristics with therapeutic courses of action).

As used herein, “protein-coding gene” means, when referring to a component of a vector, a polynucleotide that encodes a protein, other than a gene associated with the function of the vector. For example, the term protein-coding gene would encompass a polynucleotide encoding a human protein, or functional variant thereof. It is intended that the phrase “the vector comprising no other protein-coding gene” in reference to a vector means that the vector comprises a polynucleotide(s) encoding the protein of interest (e.g., AACD), but no polynucleotide encoding another protein. The phrase “the vector comprising no other protein-coding gene” does not exclude polynucleotides encoding proteins required for function of the vector, which optionally may be present, nor does the phrase exclude polynucleotides that do not encode proteins. Such vectors will include non-coding polynucleotide sequences and may include polynucleotides encoding RNA molecules (such as microRNAs). Conversely, when only certain protein-coding genes are listed, it is implied that other protein-coding genes may additionally be present.

As used herein, the term “AADC gene” refers gene that encodes an AADC polynucleotide. Introduction, administration, or other use of gene of interest should be understood to refer to any means of increasing the expression of, or increasing the activity of, a gene, gene product, or functional variant of AADC. Thus, in some embodiments, the disclosure provides methods of enhancing expression of AADC comprising introducing a polynucleotide of interest, e.g. encoding AADC, as a nucleic acid (e.g. deoxyribonucleotide (DNA) or ribonucleotide (RNA)) into a target cell as a polynucleotide (e.g. deoxyribonucleotide (DNA) or ribonucleotide (RNA)). The polynucleotide may be introduced into a cell in any of the various means known in the art, including without limitation in a viral, non-viral vector, by contacting the cell with naked polynucleotide or polynucleotide in complex with a transfection reagent, or by electroporation. Use of an AADC gene as a nucleic acid may also include indirect alteration of the expression or activity of the AADC gene, such as gene-editing of the locus encoding the endogenous gene, expression of transcription or regulatory factors, contacting cells with a small-molecule activator of the gene of interest, or use of gene-editing methods, including DNA- or RNA-based methods, to alter the expression or activity of the gene of interest as a nucleic acid. In some embodiments, the methods of the disclosure include de-repressing transcription of a gene of interest by editing regulatory regions (e.g. enhancers or promoters), altering splice sites, removing or inserting microRNA recognition sites, administering an antagomir to repress a microRNA, administering a microRNA mimetic, or any other various means of enhancing expression or activity of AADC.

As used herein, “gene product” means the product of expression of a polynucleotide sequence. For example, a protein-coding sequence is expressed by translation of the sequence into a protein gene product, or a RNA-coding sequence is expression by transcription of the DNA sequence into the corresponding RNA.

The term “vector” refers to a macromolecule or complex of molecules comprising a polynucleotide or protein to be delivered to a host cell, either in vitro or in vivo. A vector can be a modified RNA, a lipid nanoparticle (encapsulating either DNA or RNA), a transposon, an adeno-associated virus (AAV) vector, an adenovirus, a retrovirus, an integrating lentiviral vector (LVV), or a non-integrating LVV. Thus, as used herein “vectors” include naked polynucleotides used for transformation (e.g. plasmids) as well as any other composition used to deliver a polynucleotide to a cell, included vectors capable of transducing cells and vectors useful for transfection of cells. “Vector systems” refers to combinations of one, two, three, or more vectors used to delivery one, two, three, or more polynucleotides.

As used herein, the term “viral vector” refers either to a nucleic acid molecule that includes virus-derived nucleic acid elements that typically facilitate transfer of the nucleic acid molecule or integration into the genome of a cell or to a viral particle that mediates nucleic acid transfer. Viral particles will typically include various viral components and sometimes also cell components in addition to nucleic acid(s).

The term “genetic modification” refers to a permanent or transient genetic change induced in a cell following introduction of new nucleic acid (i.e., nucleic acid exogenous to the cell). Genetic change can be accomplished by incorporation of the new nucleic acid into the genome of the cell, or by transient or stable maintenance of the new nucleic acid as an extrachromosomal element. Where the cell is a eukaryotic cell, a permanent genetic change can be achieved by introduction of the nucleic acid into the genome of the cell. Suitable methods of genetic modification include viral infection, transfection, conjugation, protoplast fusion, electroporation, particle gun technology, calcium phosphate precipitation, direct microinjection, and the like.

In some embodiments, the present disclosure provides compositions and methods capable of enhancing the activity or expression of AADC in the SN. Enhancing the activity or expression of AADC in the SN is useful in prolonging the efficacy of levodopa treatment (e.g., of PD) by replacing AADC that are reduced due to degradation of dopaminergic neurons. In some embodiments, AADC is provided as a polynucleotide (e.g., an RNA, an mRNA, or a DNA polynucleotide) that encodes AADC. In some embodiments, AADC is provided as a protein.

In some embodiments, human AADC protein comprises the sequence:

```
                10 20 30 40
MNASEFRRRG KEMVDYMANY MEGIEGRQVY PDVEPGYLRP
                50 60 70 80
LIPAAAPQEP DTFEDIINDV EKIIMPGVTH WHSPYFFAYF
                90 100 110 120
PTASSYPAML ADMLCGAIGC IGFSWAASPA CTELETVMMD
              130 140 150 160
WLGKMLELPK AFLNEKAGEG GGVIQGSASE ATLVALLAAR
              170 180 190 200
TKVIHRLQAA SPELTQAAIM EKLVAYSSDQ AHSSVERAGL
              210 220 230 240
IGGVKLKAIP SDGNFAMRAS ALQEALERDK AAGLIPFFMV
              250 260 270 280
ATLGTTTCCS FDNLLEVGPI CNKEDIWLHV DAAYAGSAFI
              290 300 310 320
CPEFRHLLNG VEFADSFNFN PHKWLLVNFD CSAMWVKKRT
              330 340 350 360
DLTGAFRLDP TYLKHSHQDS GLITDYRHWQ IPLGRRFRSL
              370 380 390 400
KMWFVFRMYG VKGLQAYIRK HVQLSHEFES LVRQDPRFEI
              410 420 430 440
CVEVILGLVC FRLKGSNKVN EALLQRINSA KKIHLVPCHL
              450 460 470 480
RDKFVLRFAI CSRTVESAHV QRAWEHIKEL AADVLRAERE
```

(SEQ ID NO: 1). In some embodiments, AADC polypeptides within the scope herein capable of catalyzing the conversion of L-DOPA to dopamine. In some embodiments, AADC polypeptides within the scope herein comprise at least 70% (e.g., 70%, 75%, 80%, 85%, 90%, 95%, 100%, or ranges therebetween) sequence identity to SEQ ID NO: 1.

In some embodiments, polynucleotides encoding an AADC polypeptide comprising least 70% (e.g., 70%, 75%, 80%, 85%, 90%, 95%, 100%, or ranges therebetween) sequence identity to SEQ ID NO: 1 are provided. In some embodiments, vectors comprising a polynucleotide encoding an AADC polypeptide comprising least 70% (e.g., 70%, 75%, 80%, 85%, 90%, 95%, 100%, or ranges therebetween) sequence identity to SEQ ID NO: 1 are provided.

In some embodiments, human AADC nucleic acid comprises the sequence:

| | |
|---|---|
| 1 | aattcgggca cgagggagga cagagagcaa gtcactcccg gctgcctttt tcacctctga |
| 61 | cagagcccag acaccatgaa cgcaagtgaa ttccgaagga gagggaagga gatggtggat |
| 121 | tacgtggcca actacatgga aggcattgag ggacgccagg tctaccctga cgtggagccc |
| 181 | gggtacctgc ggccgctgat ccctgccgct gcccctcagg agccagacac gtttgaggac |
| 241 | atcatcaacg acgttgagaa gataatcatg cctggggtga cgcactggca cagcccctac |
| 301 | ttcttcgcct acttccccac tgccagctcg tacccggcca tgcttgcgga catgctgtgc |
| 361 | ggggccattg gctgcatcgg cttctcctgg gcggcaagcc cagcatgcac agagctggag |
| 421 | actgtgatga tggactggct cgggaagatg ctggaactac caaaggcatt tttgaatgag |
| 481 | aaagctggag aagggggagg agtgatccag ggaagtgcca gtgaagccac cctggtggcc |
| 541 | ctgctggccg ctcggaccaa agtgatccat cggctgcagg cagcgtcccc agagctcaca |
| 601 | caggccgcta tcatggagaa gctggtggct tactcatccg atcaggcaca ctcctcagtg |
| 661 | gaaagagctg ggttaattgg tggagtgaaa ttaaaagcca tcccctcaga tggcaacttc |
| 721 | gccatgcgtg cgtctgccct gcaggaagcc ctggagagag acaaagcggc tggcctgatt |
| 781 | cctttcttta tggttgccac cctggggacc acaacatgct gctcctttga caatctctta |
| 841 | gaagtcggtc ctatctgcaa caaggaagac atatggctgc acgttgatgc agcctacgca |
| 901 | ggcagtgcat tcatctgccc tgagttccgg caccttctga atggagtgga gtttgcagat |
| 961 | tcattcaact ttaatcccca caaatggcta ttggtgaatt ttgactgttc tgccatgtgg |
| 1021 | gtgaaaaaga gaacagactt aacgggagcc tttagactgg accccactta cctgaagcac |
| 1081 | agccatcagg attcagggct tatcactgac taccggcatt ggcagatacc actgggcaga |
| 1141 | agatttcgct ctttgaaaat gtggtttgta tttaggatgt atggagtcaa aggactgcag |
| 1201 | gcttatatcc gcaagcatgt ccagctgtcc catgagtttg agtcactggt gcgccaggat |
| 1261 | ccccgctttg aaatctgtgt ggaagtcatt ctggggcttg tctgctttcg gctaaagggt |
| 1321 | tccaacaaag tgaatgaagc tcttctgcaa agaataaaca gtgccaaaaa aatccacttg |
| 1381 | gttccatgtc acctcaggga caagtttgtc ctgcgctttg ccatctgttc tcgcacggtg |
| 1441 | gaatctgccc atgtgcagcg ggcctgggaa cacatcaaag agctggcggc cgacgtgctg |
| 1501 | cgagcagaga gggagtagga gtgaagccag ctgcaggaat caaaaattga agagagatat |
| 1561 | atctgaaaac tggaataaga agcaaataaa tatcatcctg ccttcatgga actcagctgt |
| 1621 | ctgtggcttc ccatgtcttt ctccaaagtt atccagaggg ttgtgatttt gtctgcttag |
| 1681 | tatctcatca acaaagaaat attatttgct aattaaaaag ttaatcttca tggccatagc |
| 1741 | ttttattcat tagctgtgat ttttgttgat taaaacatta tagattttca tgttcttgca |
| 1801 | gtcatcagaa gtggtaggaa agcctcactg atatattttc cagggcaatc aatgttcacg |
| 1861 | caacttgaaa ttatatctgt ggtcttcaaa ttgtcttttg tcatgtggct aaatgcctaa |
| 1921 | taaacaattc aagtgaaata ctaaaaaaaa aaaaaaaaaa aaaa |

(SEQ ID NO: 2). In some embodiments, AADC polynucleotides comprising least 70% (e.g., 70%, 75%, 80%, 85%, 90%, 95%, 100%, or ranges therebetween) sequence identity to SEQ ID NO: 2 are provided. In some embodiments, vectors comprising an AADC polynucleotide comprising least 70% (e.g., 70%, 75%, 80%, 85%, 90%, 95%, 100%, or ranges therebetween) sequence identity to SEQ ID NO: 2 are provided.

Throughout the disclosure, expression of a polynucleotide may refer to any means known in the art to increase the expression of a gene of interest (e.g., encoding an AADC). In some embodiments, the AADC is encoded in the messenger RNA (mRNA). The mRNA may be synthetic or naturally occurring. In some embodiments, the mRNA is chemically modified in various ways known in the art. For example, modified RNAs may be used, such as described in Warren, L. et al. Cell Stem Cell 7:618-30 (2010); WO2014081507A1; WO2012019168: WO2012045082: WO2012045075: WO2013052523: WO2013090648; U.S. Pat. No. 9,572,896B2; incorporated by reference in their entireties. In some embodiments, expression of the AADC gene is increased by delivery of a polynucleotide to a cell. In some embodiments, the polynucleotide encoding AADC is delivered by a viral or non-viral vector. In some embodiments, the AADC gene is encoded in the DNA polynucleotide, optionally delivered by any viral or non-viral method known in the art. In some embodiments, the disclosure provides methods comprising contacting cells with a lipid nanoparticle comprising a DNA or mRNA encoding the AADC gene. In some embodiments, the methods of the disclosure comprise contacting cells with a virus comprising a DNA or RNA (e.g., a DNA genome, a negative-sense RNA genome, a positive-sense RNA genome, or a double-stranded RNA genome) encoding the AADC gene. In some embodiments, the virus is selected from a retrovirus, adenovirus, AAV, non-integrating lentiviral vector (LVV), and an integrating LVV. In some embodiments, the cells are transfected with a plasmid. In some embodiments, the plasmid comprises a polynucleotide encoding the AADC gene. In some embodiments, the plasmid comprises a transposon comprising the AADC gene.

In some embodiments, the polynucleotides encoding the AADC gene may be codon-optimized or otherwise altered so long as the functional activity of the encoded gene is preserved. In some embodiments, the polynucleotides encode a modified or variant of the AADC gene, including truncations, insertions, deletions, or fragments, so long as the functional activity of the AADC gene is preserved.

In some embodiments, an AADC gene, polynucleotide, polypeptide, etc. is introduced into a selected cell or a selected population of cells by a vector. In some embodiments, the vector is a nucleic acid vector, such as a plasmids (e.g., DNA plasmids or RNA plasmids), transposons, cosmids, bacterial or yeast artificial chromosomes, or viral vectors. In some embodiments, the vector is a non-nucleic acid vector, such as a nanoparticle. In some embodiments, the vectors described herein comprise a peptide, such as cell-penetrating peptides or cellular internalization sequences. Cell-penetrating peptides are small peptides that are capable of translocating across plasma membranes. Exemplary cell-penetrating peptides include, but are not limited to, Antennapedia sequences, TAT, HIV-Tat, Penetratin, Antp-3A (Antp mutant), Buforin II, Transportan, MAP (model amphipathic peptide), K-FGF, Ku70), Prion, pVEC, Pep-1, SynB1, Pep-7, I-IN-1, BGSC (Bis-Guanidinium-Spermidine-Cholesterol, and BGTC (Bis-Guanidinium-Tren-Cholesterol).

Techniques in the field of recombinant genetics can be used for such recombinant expression. Basic texts disclosing general methods of recombinant genetics include Sambrook et al., Molecular Cloning, A Laboratory Manual (3rd ed. 2001); Kriegler, Gene Transfer and Expression; A Laboratory Manual (1990); and Current Protocols in Molecular Biology (Ausubel et al., eds., 1994). In some embodiments, the vectors do not contain a mammalian origin of replication. In some embodiments, the expression vector is not integrated into the genome and/or is introduced via a vector that does not contain a mammalian origin of replication.

In some cases, the expression vector(s) encodes or comprises, in addition to an AADC gene, a marker gene that facilitates identification or selection of cells that have been transfected, transduced or infected. Examples of marker genes include, but are not limited to, genes encoding fluorescent proteins, e.g., enhanced green fluorescent protein, Ds-Red (DsRed: Discosoma sp. red fluorescent protein (RFP); Bevis et al. (2002) Nat. Biotechnol. 20(11):83-87), yellow fluorescent protein, mCherry, and cyanofluorescent protein; and genes encoding proteins conferring resistance to a selection agent, e.g., a neomycin resistance gene, a puromycin resistance gene, a blasticidin resistance gene, and the like.

Suitable viral vectors include, but are not limited to, viral vectors (e.g. viral vectors based on vaccinia virus; poliovirus; adenovirus (e.g., Li et al. (1994) Invest Opthalmol Vis Sci 35:2543-2549; Borras et al. (1999) Gene Ther 6:515-524; Li and Davidson, (1995) Proc. Natl. Acad. Sci. 92:7700-7704; Sakamoto et al. (1999) Hum Gene Ther 5; 1088-1097: WO 94/12649: WO 93/03769: WO 93/19191: WO 94/28938: WO 95/11984 and WO 95/00655); adeno-associated virus (e.g., Ali et al. (1998) Hum Gene Ther 9(1):81-86, 1998, Flannery et al. (1997) Proc. Natl. Acad. Sci. 94:6916-6921; Bennett et al. (1997) Invest Opthalmol Vis Sci 38:2857-2863; Jomary et al. (1997) Gene Ther 4:683-690; Rolling et al. (1999), Hum Gene Ther 10:641-648; Ali et al. (1996) Hum Mol Genet. 5:591-594: WO 93/09239, Samulski et al. (1989) J. Vir. 63:3822-3828; Mendelson et al. (1988) Virol. 166; 154-165; and Flotte et al. (1993) Proc. Natl. Acad. Sci. 90; 10613-10617; SV40; herpes simplex virus; human immunodeficiency virus (e.g., Miyoshi et al. (1997) Proc. Natl. Acad. Sci. 94; 10319-10323; Takahashi et al. (1999) J Virol 73:7812-7816); a retroviral vector (e.g., Murine-Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. Numerous suitable expression vectors are known to those of skill in the art, and many are commercially available. The following vectors are provided by way of example; for eukaryotic cells; pXT1, pSG5 (Stratagene), pSVK3, pBPV, pMSG, pSVLSV40) (Pharmacia), and pAd (Life Technologies). However, any other vector may be used so long as it is compatible with the cells of the present disclosure.

The ability of certain viruses to infect cells or enter cells via receptor-mediated endocytosis, and express viral genes stably and efficiently have made them attractive candidates for the transfer of foreign nucleic acids into cells (e.g., mammalian cells). Viral vectors can include control sequences such as promoters for expression of the AADC polypeptide. Although many viral vectors integrate into the host cell genome, if desired, the segments that allow such integration can be removed or altered to prevent such integration. Moreover, in some embodiments, the vectors do not contain a mammalian origin of replication. Non-limiting examples of virus vectors are described below that can be used to deliver nucleic acids encoding a transcription factor into a selected cell. In some embodiments, the viral vector is derived from a replication-deficient virus.

In general, other useful viral vectors are based on non-cytopathic eukaryotic viruses in which non-essential genes have been replaced with the polypeptide of interest (AADC). Non-cytopathic viruses include certain retroviruses, the life cycle of which involves reverse transcription of genomic viral RNA into DNA with subsequent proviral integration into host cellular DNA. In general, the retroviruses are replication-deficient (e.g., capable of directing synthesis of the desired transcripts, but incapable of manufacturing an infectious particle). Such genetically altered retroviral expression vectors have general utility for the high-efficiency transduction of polynucleotide in vivo.

In some embodiments, a polynucleotide encoding an AADC polypeptide can be housed within an infective virus that has been engineered to express a specific binding ligand. The virus particle will thus bind with specificity to the cognate receptors of the target cell and deliver the contents to the cell. In some embodiments, the virus is modified to impart particular viral tropism, e.g., the virus preferentially infects neuronal cells, or more particularly dopaminergic neurons. For AAV, capsid proteins can be mutated to alter the tropism of the viral vector. For lentivirus, tropism can be modified by using different envelope proteins; this is known as “pseudotyping.”

In some embodiments, the viral vector is a retroviral vector. Retroviruses can integrate their genes into the host genome, transfer a large amount of foreign genetic material, infect a broad spectrum of species and cell types, and can be packaged in special cell-lines (Miller et al., Am. J. Clin. Oncol., 15(3):216-221, 1992). In some embodiments, a retroviral vector is altered so that it does not integrate into the host cell genome.

The recombinant retrovirus may comprise a viral polypeptide (e.g., retroviral env) to aid entry into the target cell. Such viral polypeptides are well-established in the art, for example, U.S. Pat. No. 5,449,614. The viral polypeptide may be an amphotropic viral polypeptide, for example, amphotropic env, which aids entry into cells derived from multiple species, including cells outside of the original host species. The viral polypeptide may be a xenotropic viral polypeptide that aids entry into cells outside of the original host species. In some embodiments, the viral polypeptide is an ecotropic viral polypeptide, for example, ecotropic env, which aids entry into cells of the original host species.

Examples of viral polypeptides capable of aiding entry of retroviruses into cells include, but are not limited to: MMLV amphotropic env, MMLV ecotropic env, MMLV xenotropic env, vesicular stomatitis virus-g protein (VSV-g), HIV-1 env, Gibbon Ape Leukemia Virus (GALV) env, RD114, FeLV-C, FeLV-B, MLV 10A1 env gene, and variants thereof, including chimeras. Yee et al. (1994) Methods Cell Biol, Pt A:99-1 12 (VSV-G); U.S. Pat. No. 5,449,614. In some cases, the viral polypeptide is genetically modified to promote expression or enhanced binding to a receptor.

The retroviral construct may be derived from a range of retroviruses. The retroviral construct may encode all viral polypeptides necessary for more than one cycle of replication of a specific virus. In some cases, the efficiency of viral entry is improved by the addition of other factors or other viral polypeptides. In other cases, the viral polypeptides encoded by the retroviral construct do not support more than one cycle of replication, e.g., U.S. Pat. No. 6,872,528. In such circumstances, the addition of other factors or other viral polypeptides can help facilitate viral entry.

The retroviral construct may comprise: a promoter, a multi-cloning site, and/or a resistance gene. Examples of promoters include but are not limited to CMV, SV40, EF1a, β-actin; retroviral LTR promoters, and inducible promoters. The retroviral construct may also comprise a packaging signal (e.g., a packaging signal derived from the MFG vector; a psi packaging signal). Examples of some retroviral constructs known in the art include but are not limited to: pMX, pBabeX or derivatives thereof. Onishi et al. (1996) Experimental Hematology, 24:324-329. In some cases, the retroviral construct is a self-inactivating lentiviral vector (SIN) vector. Miyoshi et al. (1998) J. Virol 72(10):8150-8157. In some cases, the retroviral construct is LL-CG, LS-CG, CL-CG, CS-CG, CLG or MFG. Miyoshi et al. (1998) J. Virol 72(10):8150-8157; Onishi et al. (1996) Experimental Hematology, 24:324-329; Riviere et al. (1995) Proc. Natl. Acad. Sci., 92:6733-6737.

A retroviral vector can be constructed by inserting a nucleic acid (e.g., one encoding an AADC polypeptide) into the viral genome in the place of some viral sequences to produce a virus that is replication-defective. To produce virions, a packaging cell line containing the gag, pol, and env genes, but without the LTR and packaging components, is constructed (Mann et al., Cell 33:153-159, 1983). When a recombinant plasmid containing a cDNA, together with the retroviral LTR and packaging sequences is introduced into a special cell line (e.g., by calcium phosphate precipitation), the packaging sequence allows the RNA transcript of the recombinant plasmid to be packaged into viral particles, which are then secreted into the culture media (Nicolas and Rubinstein, In: Vectors; A survey of molecular cloning vectors and their uses, Rodriguez and Denhardt, eds., Stoneham; Butterworth, pp. 494-513, 1988; Temin, In: Gene Transfer, Kucherlapati (ed.), New York: Plenum Press, pp. 149-188, 1986; Mann et al., Cell, 33:153-159, 1983). The media containing the recombinant retroviruses is then collected, optionally concentrated, and used for gene transfer. Retroviral vectors are able to infect a broad variety of cell types. However, integration and stable expression typically involves the division of host cells (Paskind et al., Virology, 67:242-248, 1975).

In some embodiments, the viral vector is an adenoviral vector. The genetic organization of adenovirus includes an approximate 36 kb, linear, double-stranded DNA virus, which allows substitution of large pieces of adenoviral DNA with foreign sequences up to 7 kb (Grunhaus et al., Seminar in Virology 200(2):535-546, 1992)). An AADC polypeptide or polynucleotide may be introduced into the cell using adenovirus assisted transfection. Increased transfection efficiencies have been reported in cell systems using adenovirus coupled systems (Kelleher and Vos, Biotechniques, 17(6): 1110-7, 1994; Cotten et al., Proc Natl Acad Sci USA, 89(13):6094-6098, 1992; Curiel, Nat Immun, 13 (2-3): 141-64, 1994.).

In some embodiments, the viral vector is an AAV vector. AAV is an attractive vector system as it has a low frequency of integration and it can infect non-dividing cells, thus making it useful for delivery of polynucleotides into mammalian cells, for example, in tissue culture (Muzyczka, Curr Top Microbiol Immunol, 158:97-129, 1992) or in vivo. Details concerning the generation and use of rAAV vectors are described in U.S. Pat. Nos. 5,139,941 and 4,797,368, each incorporated herein by reference in its entirety.

AAV is a replication-deficient parvovirus, the single-stranded DNA genome of which is about 4.7 kb in length including two 145 nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077: the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). The sequence of the AAV rh.74 genome is provided in U.S. Pat. No. 9,434,928, incorporated herein by reference. Cis-acting sequences directing viral DNA replication (rep), encapsidation/packaging and host cell chromosome integration are contained within the AAV ITRs. Three AAV promoters (named p5, p19, and p40) for their relative map locations) drive the expression of the two AAV internal open reading frames encoding rep and cap genes. The two rep promoters (p5 and pi 9), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus, making cold preservation of AAV less critical. AAV may even be lyophilized. Finally, AAV-infected cells are not resistant to superinfection. The AAV vectors of the disclosure include self-complementary, duplexed AAV vectors, synthetic ITRs, and/or AAV vectors with increased packaging capacity. Illustrative methods are provided in U.S. Pat. Nos. 8,784,799; 8,999,678; 9,169,494; 9,447,433; and 9,783,824, each of which is incorporated by reference in its entirety.

AAV DNA in the rAAV genomes may be from any AAV serotype for which a recombinant virus can be derived including, but not limited to, AAV serotypes AAV-1. AAV-2, AAV-3, AAV-4, AAV-5, AAV-6, AAV-7, AAV-8, AAV-9, AAV-10, AAV-11, AAV-12, AAV-13 and AAV rh74. Production of pseudotyped rAAV is disclosed in, for example, WO 01/83692. Other types of rAAV variants, for example rAAV with capsid mutations, are also contemplated. See, for example, Marsic et al., Mol. Therapy, 22): 1900-09 (2014). The nucleotide sequences of the genomes of various AAV serotypes are known in the art. AAV vectors of the present disclosure include AAV vectors of serotypes AAV1, AAV2, AAV4, AAV5, AAV6, AAV7, AAV8, AAV9, AAV10, AAV39, AAV43, AAV.rh74, and AAV.rh8. Illustrative AAV vectors are provided in U.S. Pat. No. 7,105,345; U.S. Ser. No. 15/782,980; U.S. Pat. Nos. 7,259,151; 6,962,815; 7,718,424; 6,984,517; 7,718,424; 6,156,303; 8,524,446; 7,790,449; 7,906,111; 9,737,618; U.S. application Ser. No. 15/433,322; U.S. Pat. No. 7,198,951, each of which is incorporated by reference in its entirety.

In some embodiments, the AAV expression vector is pseudotyped to enhance targeting. To promote gene transfer and sustain expression in fibroblasts, AAV5, AAV7, and AAV8, may be used. In some cases, the AAV2 genome is packaged into the capsid of producing pseudotyped vectors AAV2/5, AAV2/7, and AAV2/8 respectively, as described in Balaji et al. J Surg Res. 184:691-98 (2013). In some embodiments, AAV1, AAV6, or AAV9 is used, and in some embodiments, the AAV is engineered, as described in Asokari et al. Hum Gene Ther. 24:906-13 (2013); Pozsgai et al. Mol Ther. 25:855-69 (2017); Kotterman et al. Nature Reviews Genetics 15:445-51 (2014); and US20160340393A1 to Schaffer et al. In some embodiments, the viral vector is AAV engineered to increase target cell infectivity as described in US20180066285A1.

In some embodiments, the AAV vectors of the disclosure comprises a modified capsid, in particular as capsid engineered to enhance or promote in vivo or ex vivo transduction of cells, or more particularly neuronal cells, or dopaminergic neurons in particular; or that evade the subject's immune system; or that have improved biodistribution. Illustrative AAV capsids are provided in U.S. Pat. Nos. 7,867,484; 9,233,131; 10,046,016; WO 2016/133917: WO 2018/222503; and WO 20019/060454, each of which is incorporated by reference in its entirety. More particularly, the AAV vectors of the disclosure, optionally AAV2-based vectors, may comprise in their capsid proteins one or more substitutions selected from E67A, S207G, V2291, A490T, N551S, A58IT, and I698V. In some embodiments, the AAV vectors of the disclosure comprise the AAV-A2 capsid and/or serotype, which is described in WO 2018/222503. In some embodiments, the AAV capsid comprises an insertion in the GH loop of the capsid protein, such as NKIQRTD (SEQ ID NO: 65) or NKTTNKD (SEQ ID NO: 66). It will be appreciated that these substitutions and insertions may be combined together to generate various capsid proteins useful in the present disclosure.

In some embodiments, the viral vector is a lentiviral vector. Lentiviruses are complex retroviruses, which, in addition to the common retroviral genes gag, pol, and env, contain other genes with regulatory or structural function. Information on lentiviral vectors is available, for example, in Naldini et al., Science 272(5259):263-267, 1996; Zufferey et al., Nat Biotechnol 15(9): 871-875, 1997; Blomer et al., J Virol. 71(9):6641-6649, 1997; U.S. Pat. Nos. 6,013,516 and 5,994,136, each of which is incorporated herein by reference in its entirety. Some examples of lentivirus include the Human Immunodeficiency Viruses: HIV-1, HIV-2 and the Simian Immunodeficiency Virus: SIV. Lentiviral vectors have been generated by attenuating the HIV virulence genes, for example, the genes env, vif, vpr, vpu and nef are deleted to make the vector biologically safe. The lentivirus employed can also be replication and/or integration defective.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, which is incorporated herein by reference in its entirety. Those of skill in the art can target the recombinant virus by linkage of the envelope protein with an antibody or a particular ligand for targeting to a receptor of a particular cell type. For example, a target-specific vector can be generated by inserting a nucleic acid segment (including a regulatory region) comprising AADC into the viral vector, along with another gene that encodes a ligand for a receptor on a specific target cell type.

Lentiviral vectors are known in the art, see Naldini et al., (1996 and 1998); Zufferey et al., (1997); Dull et al., 1998, U.S. Pat. Nos. 6,013,516; and 5,994,136 all incorporated herein by reference. In general, these vectors are plasmid-based or virus-based, and are configured to carry the essential sequences for incorporating foreign nucleic acid, for selection and for transfer of the nucleic acid into a host cell. In some cases, a lentiviral vector is introduced into a cell concurrently with one or more lentiviral packaging plasmids, which may include, without limitation, pMD2.G, pRSV-rev, pMDLG-pRRE, and pRRL-GOI. Introduction of a lentiviral vector alone or in combination with lentiviral packaging plasmids into a cell may cause the lentiviral vector to be packaged into a lentiviral particle. In some embodiments, the lentiviral vector is a non-integrating lentiviral (NIL) vector. Illustrative methods for generating NIL vectors, such as the D64V substitution in the integrase gene, are provided in U.S. Pat. No. 8,119,119.

Virus vector plasmids (or constructs), include: pMXs, pMxs-IB, pMXs-puro, pMXs-neo (pMXs-IB is a vector carrying the blasticidin-resistant gene instead of the puromycin-resistant gene of pMXs-puro) Kimatura et al. (2003) Experimental Hematology 31: 1007-1014; MFG Riviere et al. (1995) Proc. Natl. Acad. Sci., 92:6733-6737; pBabePuro; Morgenstern et al. (1990) Nucleic Acids Research 18:3587-3596; LL-CG, CL-CG, CS-CG, CLG Miyoshi et al. (1998) J. Vir. 72:8150-8157 and the like as the retrovirus system, and pAdexl Kanegae et al. (1995) Nucleic Acids Research 23:3816-3821 and the like as the adenovirus system. In exemplary embodiments, the retroviral construct comprises blasticidin (e.g., pMXs-IB), puromycin (e.g., pMXs-puro, pBabePuro), or neomycin (e.g., pMXs-neo). Morgenstern et al. (1990) Nucleic Acids Research 18:3587-3596.

In some embodiments, the viral vector or plasmid comprises a transposon or a transposable element comprising a polynucle otide encoding a AADC polypeptide. Delivery of polynucleotides via DNA transposons, such as piggy Bac and Sleeping Beauty, offers advantages in ease of use, ability to delivery larger cargo, speed to clinic, and cost of production. The piggy Bac DNA transposon, in particular, offers potential advantages in giving long-term, high-level and stable expression of polynucleotides, and in being significantly less mutagenic, being non-oncogenic and being fully reversible.

In some embodiments, an AADC gene is introduced as an RNA molecule, which is translated to protein within the cell's cytoplasm. For example, the AADC gene is translated from introduced RNA molecules that have the open reading frame (ORF) for the polypeptide flanked by a 5′ untranslated region (UTR) containing a translational initiation signal (e.g., a strong Kozak translational initiation signal) and a 3′ untranslated region terminating with an oligo(dT) sequence for templated addition of a polyA tail. Such RNA molecules do not have the promoter sequences employed in most expression vectors and expression cassettes. The RNA molecules can be introduced into the selected cells by a variety of techniques, including electroporation or by endocytosis of the RNA complexed with a cationic vehicle. See, e.g., Warren et al., Cell Stem Cell 7: 618-30 (2010), incorporated herein by reference in its entirety. Protein translation can persist for several days, especially when the RNA molecules are stabilized by incorporation of modified ribonucleotides. For example, incorporation of 5-methylcytidine (5mC) for cytidine and/or pseudouridine (psi) for uridine can improve the half-life of the introduced RNA in vivo, and lead to increased protein translation. If high levels of expression are desired, or expression for more than a few days is desired, the RNA can be introduced repeatedly into the selected cells. The RNA encoding the protein can also include a 5′ cap, a nuclear localization signal, or a combination thereof. See, e.g., Warren et al., Cell Stem Cell 7: 618-30 (2010). Such RNA molecules can be made, for example, by in vitro transcription of a template for the AADC polynucleotide using a ribonucleoside blend that includes a 3′-O-Me-m7G(5′)ppp(5′)G ARCA cap analog, adenosine triphosphate and guanosine triphosphate, 5-methylcytidine triphosphate and pseudouridine triphosphate. The RNA molecules can also be treated with phosphatase to reduce cytotoxicity. The RNA can be introduced alone or with a microRNA (e.g., for Oct4 expression, miRNA-302), which can be an inducer of endogenous polypeptide expression. The microRNA functions as a structural RNA that does not encode a protein. Hence, no translation is needed for microRNA to perform its function. The microRNA can be introduced directly into cells, for example, in a delivery vehicle such as a liposome, microvesicle, or exosome. Alternatively, the microRNA can be expressed from an expression cassette or expression vector that has been introduced into a cell or a cell population.

In certain embodiments, the vector comprises lipid particles as described in Kanasty R. Delivery materials for siRNA therapeutics Nat Mater. 12(11):967-77 (2013), which is hereby incorporated by reference. In some embodiments, the lipid-based vector is a lipid nanoparticle, which is a lipid particle between about 1 and about 100 nanometers in size. In some embodiments, the lipid-based vector is a lipid or liposome. Liposomes are artificial spherical vesicles comprising a lipid bilayer. In some embodiments, the lipid-based vector is a small nucleic acid-lipid particle (SNALP). SNALPs comprise small (less than 200 nm in diameter) lipid-based nanoparticles that encapsulate a nucleic acid. In some embodiments, the SNALP is useful for delivery of an RNA molecule such as siRNA. In some embodiments. SNALP formulations deliver nucleic acids to a particular tissue in a subject.

In some embodiments, the one or more polynucleotides are delivered via polymeric vectors. In some embodiments, the polymeric vector is a polymer or polymerosome. Polymers encompass any long repeating chain of monomers and include, for example, linear polymers, branched polymers, dendrimers, and polysaccharides. Linear polymers comprise a single line of monomers, whereas branched polymers include side chains of monomers. Dendrimers are also branched molecules, which are arranged symmetrically around the core of the molecule. Polysaccharides are polymeric carbohydrate molecules, and are made up of long monosaccharide units linked together. Polymersomes are artificial vesicles made up of synthetic amphiphilic copolymers that form a vesicle membrane, and may have a hollow or aqueous core within the vesicle membrane. Various polymer-based systems can be adapted as a vehicle for administering DNA or RNA encoding the AADC polypeptide. Exemplary polymeric materials include poly(D,L-lactic acid-co-glycolic acid) (PLGA), poly(caprolactone) (PCL), ethylene vinyl acetate polymer (EVA), poly(lactic acid) (PLA), poly(L-lactic acid) (PLLA), poly(glycolic acid) (PGA), poly(L-lactic acid-co-glycolic acid) (PLLGA), poly(D,L-lactide) (PDLA), poly(L-lactide) (PLLA). PLGA-b-poly(ethylene glycol)-PLGA (PLGA-bPEG-PLGA), PLLA-bPEG-PLLA. PLGA-PEG-maleimide (PLGA-PEG-mal), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) (polyacrylic acids), and copolymers and mixtures thereof, polydioxanone and its copolymers, polyhydroxyalkanoates, polypropylene fumarate), polyoxymethylene, poloxamers, poly(ortho)esters, poly(butyric acid), poly(valeric acid), poly(lactide-co-caprolactone), trimethylene carbonate, polyvinylpyrrolidone, polyorthoesters, polyphosphazenes, Poly([beta]-amino esters (PBAE), and polyphosphoesters, and blends and/or block copolymers of two or more such polymers. Polymer-based systems may also include Cyclodextrin polymer (CDP)-based nanoparticles such as, for example, CDP-admantane (AD)-PEG conjugates and CDP-AD-PEG-transferrin conjugates. Exemplary polymeric particle systems for delivery of drugs, including nucleic acids, include those described in U.S. Pat. Nos. 5,543,158, 6,007,845, 6,254,890, 6,998,115, 7,727,969, 7,427,394, 8,323,698, 8,071,082, 8,105,652, US 2008/0268063, US 2009/0298710, US 2010/0303723, US 2011/0027172, US 2011/0065807, US 2012/0156135, US 2014/0093575, WO 2013/090861, each of which are hereby incorporated by reference in its entirety.

In some embodiments, a nucleic acid encoding an AADC polypeptide can be operably linked to a promoter and/or enhancer to facilitate expression of the AADC. Depending on the host/vector system utilized, any of a number of suitable transcription and translation control elements, including constitutive and inducible promoters, transcription enhancer elements, transcription terminators, etc. may be used in the expression vector (e.g., Bitter et al. (1987) Methods in Enzymology, 153:516-544). Non-limiting examples of suitable eukaryotic promoters (promoters functional in a eukaryotic cell) include CMV, CMV immediate early, HSV thymidine kinase, early and late SV40, long terminal repeats (LTRs) from retrovirus, and mouse metallothionein-I. In some embodiments, promoters that are capable of conferring neuronal-specific expression will be used.

Various techniques may be employed for introducing nucleic acid molecules of the disclosure into cells. Such techniques include transfection of nucleic acid molecule-calcium phosphate precipitates, transfection of nucleic acid molecules associated with DEAE, transfection or infection with the foregoing viruses including the nucleic acid molecule of interest, liposome-mediated transfection, and the like. Other examples include: N-TER Nanoparticle Transfection System by Sigma-Aldrich, FECTOFLY transfection reagents for insect cells by Polyplus Transfection, Polyethylenimine “Max” by Polysciences, Inc., Unique, Non-Viral Transfection Tool by Cosmo Bio Co., Ltd., LIPOFECTAMINE LTX Transfection Reagent by Invitrogen, SATISFECTION Transfection Reagent by Stratagene, LIPOFECTAMINE Transfection Reagent by Invitrogen, FUGENE HD Transfection Reagent by Roche Applied Science, GMP compliant IN VIVO-JETPEI transfection reagent by Polyplus Transfection, and Insect GENEJUICE Transfection Reagent by Novagen.

### EXPERIMENTAL

### Results

### Slow Loss of MCI Function Induced Metabolic Reprogramming

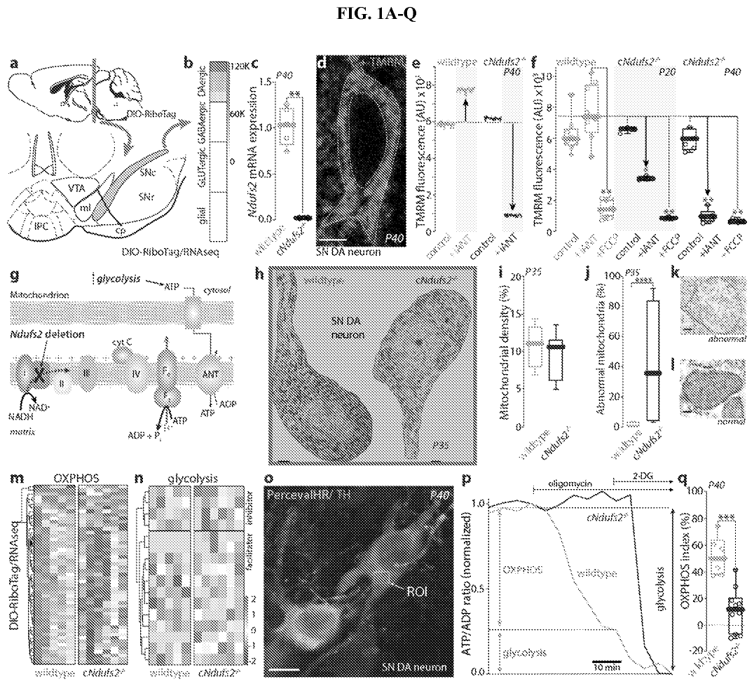

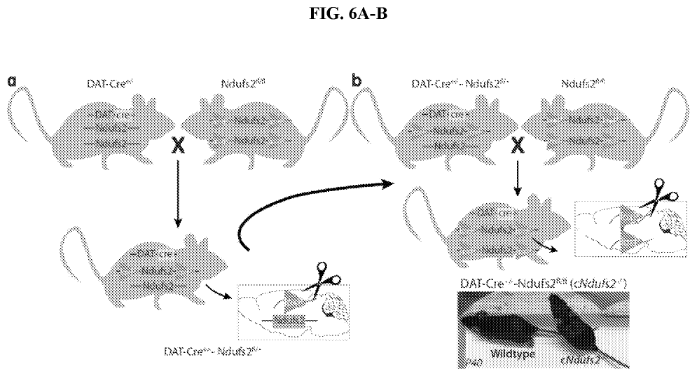

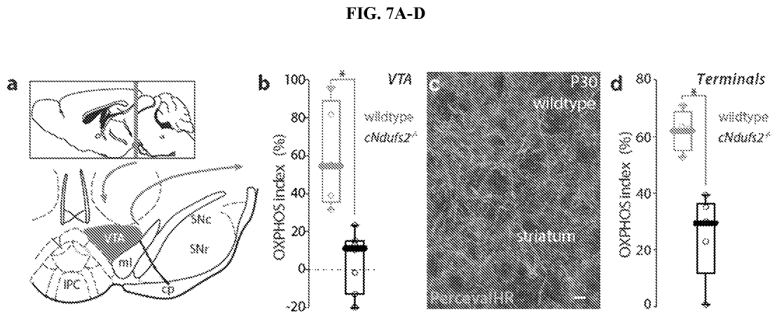

To delete Ndufs2 specifically from dopaminergic neurons, mice in which the gene was floxed (Ndufs fl/fl) were crossed with ones expressing Cre recombinase (Cre) under control of the promoter for the dopamine transporter (DAT) (DAT-Cre+/−) (FIG. 6, Table 1) (Refs. 9, 11, 12; incorporated by reference in their entireties). The efficacy of the Ndufs2 deletion was confirmed by isolating actively transcribed mRNA from SN dopaminergic neurons using the RiboTag method (Ref. 13: incorporated by reference in its entirety) and then performing a quantitative, reverse transcriptase polymerase chain reaction (qRT-PCR) assay (FIG. 1a-c). Through weaning (˜P20), conditional Ndufs2 knockout (cNdufs2−/−) mice were normal in appearance and gross motor behavior. As MCI proteins commonly have lifetimes of 20-40 days 14, the absence of a phenotype in this early postnatal period was not surprising. However, between P20 and P30, mitochondria in both ventral tegmental area (VTA) (FIG. 7) and SN dopaminergic neurons became net consumers, rather than producers, of adenosine triphosphate (ATP). This shift was evident in the sensitivity of the inner mitochondrial membrane (IMM) potential measured with the potentiometric dye tetramethylrhodamine (TMRM) to blockade of the adenine nucleotide transporter (ANT) (FIGS. 1d-f). In ex vivo brain slices from wildtype mice, dopaminergic neuron IMM potential rose with ANT blockade, as the dissipative activity of complex V presumably was inhibited by slowing of ATP transport out of the matrix (FIGS. 1e, f). However, in cNdufs2−/− dopaminergic neurons, ANT blockade caused the IMM potential to collapse, as it prevented mitochondria from importing ATP and running complex V in reverse (FIGS. 1e-g).

TABLE 1
Neuronal markers in wildtype mice at P40

| | |
|---|---|
| Gene | Description |

Dopaminergic markers

| | |
|---|---|
| Th | tyrosine hydroxylase |
| Slc6a3 | solute carrier family 6 (neurotransmitter |
| | transporter, dopamine), member 3 |
| Ddc | dopa decarboxylase |
| Slc18a2 | solute carrier family 18 (vesicular monoamine), member 2 |

Gabaergic markers

| | |
|---|---|
| Gabra2 | gamma-aminobutyric acid (GABA) A receptor, subunit alpha 2 |
| Gabrr3 | gamma-aminobutyric acid (GABA) receptor, rho 3 |
| Gad1 | glutamate decarboxylase 1 |
| Gabrg1 | gamma-aminobutyric acid (GABA) A receptor, subunit gamma 1 |
| Gabrg3 | gamma-aminobutyric acid (GABA) A receptor, subunit gamma 3 |

Glutamatergic markers

| | |
|---|---|
| Slc17a7 | solute carrier family 17 (sodium-dependent |
| | inorganic phosphate cotransporter), member 7 |
| Slc17a6 | solute carrier family 17 (sodium-dependent |
| | inorganic phosphate cotransporter), member 6 |
| Grin1 | glutamate receptor, ionotropic, NMDA1 (zeta 1) |
| Grm6 | glutamate receptor, metabotropic 6 |
| Grik1 | glutamate receptor, ionotropic, kainate 1 |

Glial markers

| | |
|---|---|
| Aldh1l1 | aldehyde dehydrogenase 1 family, member L1 |
| S100b | S100 protein, beta polypeptide, neural |
| Aqp4 | aquaporin 4 |
| Gfap | glial fibrillary acidic protein |
| Fgfr3 | fibroblast growth factor receptor 3 |

To determine if the mitochondrial OXPHOS deficit triggered changes in mitochondrial morphology or density, SN dopaminergic neurons were retrogradely labeled by injecting Fluoro-Gold into the striatum and then examined using transmission electron microscopy. Consistent with the proposition that mitochondria were maintaining their membrane potential, somatic mitochondrial density was normal in cNdufs2^{-/-} dopaminergic neurons (FIGS. 1h, i). Thus, mitochondria were not sufficiently depolarized to induce mitophagy (Ref. 15; incorporated by reference in its entirety). However, in many cNdufs2^{-/-} dopaminergic neurons, mitochondrial cristae structure was altered (FIGS. 1j-l), consistent with the down-regulation of electron transport chain genes (Ref. 16; incorporated by reference in its entirety). This modest structural change stands in contrast to the gross mitochondrial pathology and turnover seen in dopaminergic neurons in the MitoPark mouse, where mitochondrial gene transcription is widely disrupted (Ref. 17; incorporated by reference in its entirety).

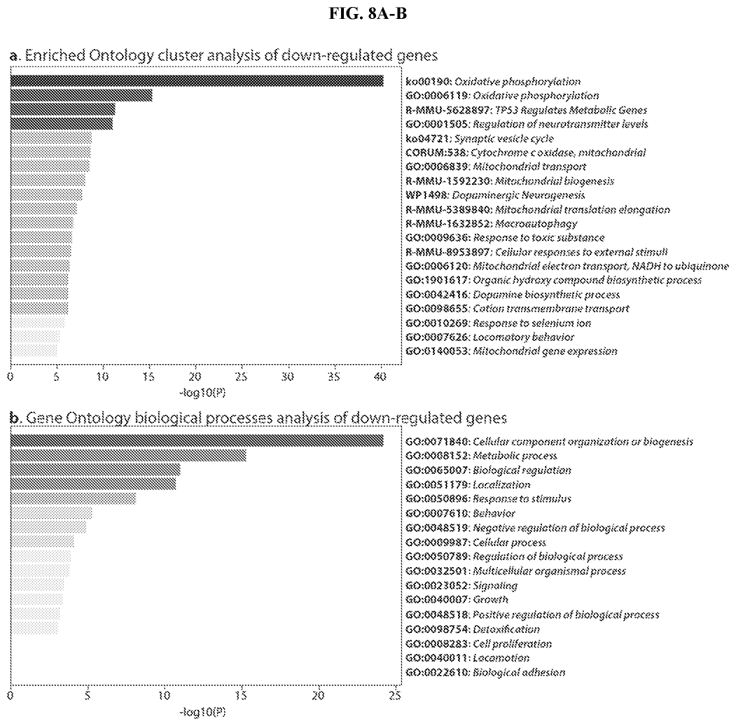

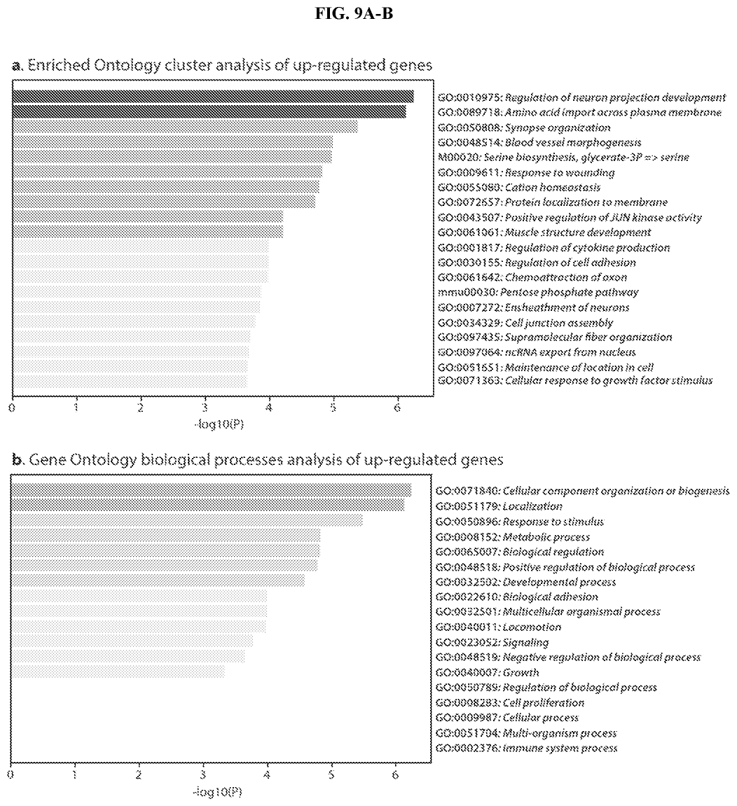

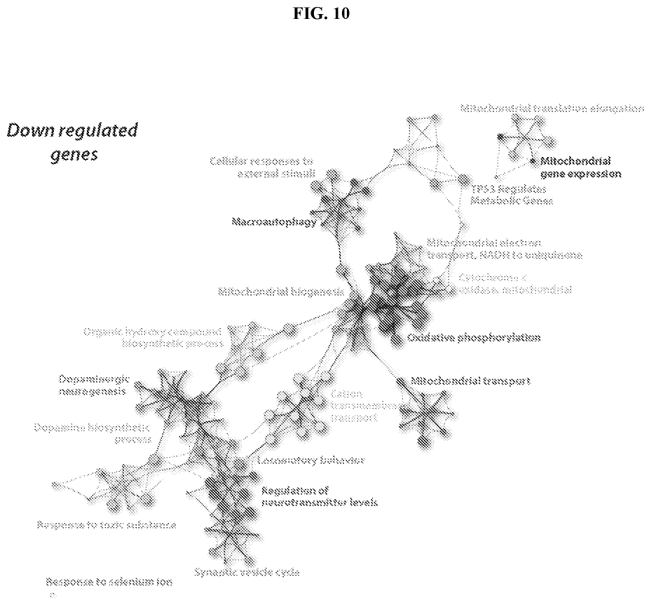

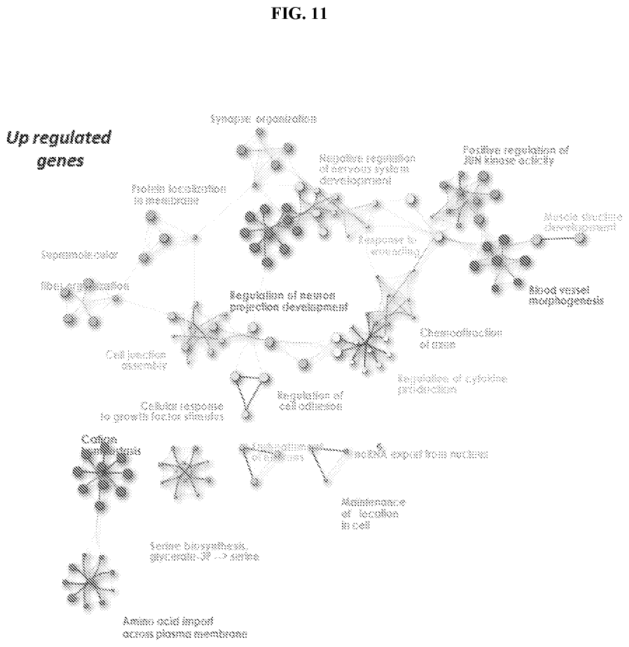

To get a better picture of the epigenetic changes induced by disruption of mitochondrial OXPHOS, mRNA from wildtype and cNdufs2−/− SN dopaminergic neurons was isolated using the RiboTag approach and then sequenced13. This analysis revealed a dramatic metabolic reprogramming—a Warburg-like effect—in cNdufs2−/− dopaminergic neurons. That is, there was an up-regulation of genes coding for proteins promoting glycolysis and down-regulation of those associated with OXPHOS (FIGS. 1m, n; FIGS. 8 to 11; Tables 2 and 3). Genes coding for inhibitors of glycolysis also were down-regulated (e.g., TP53-inducible glycolysis and apoptosis regulator) (FIGS. 8 to 11; Tables 2 and 3). Indeed, measurement of cytosolic ATP/ADP (adenosine diphosphate) ratio using the genetically encoded sensor PercevalHR (Ref. 18; incorporated by reference in its entirety), revealed that, in contrast to wildtype dopaminergic neurons where inhibition of mitochondrial complex V with oligomycin caused a precipitous drop in ATP levels, in cNdufs21-dopaminergic neurons this ratio fell only in response to inhibition of glycolysis (FIGS. 1o-q, FIG. 7).

TABLE 2
Oxidative phosphorylation - P40

| | | Fold Change | | |
|---|---|---|---|---|
| Gene | Description | (log₂) | p Value | FDR |
| Sdha | succinate dehydrogenase complex, subunit A, | 0.39928282 | 0.001122 | 0.020841 |
| | flavoprotein (Fp) | | | |
| Cox6a2 | cytochrome c oxidase subunit 6A2 | 5.94618643 | 2.06E−09 | 1.81E−07 |
| Atp6v0b | ATPase, H+ transporting, lysosomal V0 subunit B | −0.2781173 | 0.002088 | 0.033120 |
| Cox6b2 | cytochrome c oxidase subunit 6B2 | −1.6969963 | 2.69E−15 | 7.88E−13 |
| Cox7a1 | cytochrome c oxidase subunit 7A1 | −1.9788422 | 2.65E−06 | 0.000129 |
| Ndufs2 | NADH:ubiquinone oxidoreductase core subunit S2 | −1.8238989 | 4.74E−25 | 5.03E−22 |
| Ndufs5 | NADH:ubiquinone oxidoreductase core subunit S5 | −0.3562319 | 0.000566 | 0.012180 |
| Ppa1 | pyrophosphatase (inorganic) 1 | −0.6121229 | 9.10E−05 | 0.002722 |
| Atp5d | ATP synthase, H+ transporting, mitochondrial F1 | −0.4231161 | 1.79E−07 | 1.18E−05 |
| | complex, delta subunit | | | |
| Ndufa7 | NADH:ubiquinone oxidoreductase subunit A7 | −0.4872341 | 2.86E−06 | 0.000139 |
| Atp6v1f | ATPase, H+ transporting, lysosomal V1 subunit F | −0.3889854 | 5.81E−05 | 0.001914 |
| Atp6v0c | ATPase, H+ transporting, lysosomal V0 subunit C | −0.6078364 | 1.42E−08 | 1.10E−06 |
| Ndufa1 | NADH:ubiquinone oxidoreductase subunit A1 | −0.3919798 | 0.000788 | 0.016130 |
| Cox17 | cytochrome c oxidase assembly protein 17, copper | −0.4207554 | 0.000518 | 0.011345 |
| | chaperone | | | |
| Ndufb2 | NADH:ubiquinone oxidoreductase subunit B2 | −0.7363380 | 1.04E−10 | 1.06E−08 |
| Atp5j2 | ATP synthase, H+ transporting, mitochondrial F0 | −0.4855929 | 6.25E−07 | 3.55E−05 |
| | complex, subunit F2 | | | |
| Atp5k | ATP synthase, H+ transporting, mitochondrial F1F0 | −0.8197073 | 4.86E−11 | 5.04E−09 |
| | complex, subunit E | | | |
| Uqcrh | ubiquinol-cytochrome c reductase hinge protein | −0.5054854 | 5.83E−08 | 4.15E−06 |
| Ndufa5 | NADH:ubiquinone oxidoreductase subunit A5 | −0.3452814 | 0.000653 | 0.013633 |
| Cox6a1 | cytochrome c oxidase subunit 6A1 | −0.3947356 | 8.10E−05 | 0.002479 |
| Ndufa6 | NADH:ubiquinone oxidoreductase subunit A6 | −0.4932714 | 3.27E−05 | 0.001176 |
| Ndufa2 | NADH:ubiquinone oxidoreductase subunit A2 | −0.3383293 | 0.001206 | 0.021983 |
| Ndufa13 | NADH:ubiquinone oxidoreductase subunit A13 | −0.3561455 | 4.84E−06 | 0.000226 |
| Ndufb11 | NADH.ubiquinone oxidoreductase subunit B11 | −0.4591074 | 7.69E−06 | 0.000341 |
| Cox7a2 | cytochrome c oxidase subunit 7A2 | −0.6427115 | 3.27E−11 | 3.56E−09 |
| Ndufb10 | NADH:ubiquinone oxidoreductase subunit B10 | −0.3387047 | 0.000238 | 0.006087 |
| Ndufs6 | NADH:ubiquinone oxidoreductase core subunit S6 | −0.3368072 | 8.06E−05 | 0.002479 |
| Ndufb7 | NADH:ubiquinone oxidoreductase subunit B7 | −0.3929082 | 3.04E−05 | 0.001113 |
| Cox8a | cytochrome c oxidase subunit 8A | −0.5297268 | 1.19E−10 | 1.20E−08 |
| Uqcrq | ubiquinol-cytochrome c reductase, complex III subunit | −0.4983119 | 7.61E−12 | 8.75E−10 |
| | VII | | | |
| Uqcc2 | ubiquinol-cytochrome c reductase complex assembly | −0.4703206 | 3.07E−05 | 0.001120 |
| | factor 2 | | | |
| Atp6v1g2 | ATPase, H+ transporting, lysosomal V1 subunit G2 | −0.3582802 | 0.000195 | 0.005113 |
| Atp5e | ATP synthase, H+ transporting, mitochondrial F1 | −0.6028318 | 2.01E−06 | 0.000101 |
| | complex, epsilon subunit | | | |
| Cox7b | cytochrome c oxidase subunit 7B | −0.5298796 | 8.87E−07 | 4.87E−05 |
| Ndufc1 | NADH:ubiquinone oxidoreductase subunit C1 | −0.7317227 | 1.03E−08 | 8.18E−07 |
| Cox6b1 | cytochrome c oxidase, subunit 6B1 | −0.3854526 | 2.58E−05 | 0.000976 |
| Ndufv3 | NADH:ubiquinone oxidoreductase core subunit V3 | −0.4419430 | 6.62E−06 | 0.000300 |
| Uqcr10 | ubiquinol-cytochrome c reductase, complex III subunit X | −0.6643168 | 2.15E−09 | 1.86E−07 |
| Cox6c | cytochrome c oxidase subunit 6C | −0.6149164 | 2.02E−09 | 1.79E−07 |
| Ndufa3 | NADH:ubiquinone oxidoreductase subunit A3 | −0.6940632 | 1.21E−10 | 1.20E−08 |
| Sdhaf4 | succinate dehydrogenase complex assembly factor 4 | −0.7501573 | 9.51E−06 | 0.000404 |
| Ndufa11 | NADH:ubiquinone oxidoreductase subunit A11 | −0.3671749 | 0.000108 | 0.003167 |
| Atp5g3 | ATP synthase, H+ transporting, mitochondrial F0 | −0.3517200 | 0.000261 | 0.006586 |
| | complex, subunit C3 (subunit 9) | | | |
| Atp5o | ATP synthase, H+ transporting, mitochondrial F1 | −0.3114885 | 0.000747 | 0.015408 |
| | complex, O subunit | | | |
| Uqcr11 | ubiquinol-cytochrome c reductase, complex III subunit XI | −0.4929112 | 5.10E−07 | 2.98E−05 |
| Ndufb8 | NADH:ubiquinone oxidoreductase subunit B8 | −0.2813261 | 0.000292 | 0.007195 |
| Cox5a | cytochrome c oxidase subunit 5A | −0.3969596 | 0.000169 | 0.004546 |
| Ndufb5 | NADH:ubiquinone oxidoreductase subunit B5 | −0.2852090 | 0.001772 | 0.029309 |
| Chchd10 | coiled-coil-helix-coiled-coil-helix domain containing 10 | −0.2659468 | 0.000417 | 0.009658 |
| Ndufb3 | NADH:ubiquinone oxidoreductase subunit B3 | −0.5783289 | 5.31E−07 | 3.08E−05 |
| Atp5h | ATP synthase, H+ transporting, mitochondrial F0 | −0.3940754 | 1.11E−05 | 0.000458 |
| | complex, subunit D | | | |

TABLE 3
Glycolysis - P40

| | | Fold Change | | |
|---|---|---|---|---|
| Gene | Description | (log₂) | p Value | FDR |
| Mpc2 | mitochondrial pyruvate carrier 2 | −0.4164967 | 9.52E−05 | 0.002835 |
| Gucy2c | guanylate cyclase 2c | −0.9767479 | 0.000122 | 0.003449 |
| Mif | macrophage migration inhibitory factor | −0.6147396 | 1.27E−06 | 6.61E−05 |
| | (glycosylation-inhibiting factor) | | | |
| Tigar | Tp53 induced glycolysis regulatory phosphatase | −1.1590881 | 7.41E−06 | 0.000331 |
| Hkdc1 | hexokinase domain containing 1 | 8.60638557 | 0.000709 | 0.014665 |
| Adh7 | alcohol dehydrogenase 7 (class IV), mu or sigma | 8.66940222 | 0.000636 | 0.013430 |
| | polypeptide | | | |
| Pmm1 | phosphomannomutase 1 | 0.27255278 | 0.003360 | 0.046772 |
| Got1 | glutamic-oxaloacetic transaminase 1, soluble | 0.33413523 | 0.002401 | 0.036521 |
| Pgm2 | phosphoglucomutase 2 | 0.79751159 | 7.83E−05 | 0.002431 |
| Aldoc | aldolase C, fructose-bisphosphate | 0.57502795 | 1.08E−09 | 9.82E−08 |
| Prmt1 | protein arginine N-methyltransferase 1 | 0.49121732 | 0.000398 | 0.009264 |
| Gpd1 | glycerol-3-phosphate dehydrogenase 1 (soluble) | 2.09724786 | 0.002464 | 0.037151 |
| Car10 | carbonic anhydrase 10 | 1.46600098 | 0.001210 | 0.022026 |
| Slc37a1 | solute carrier family 37 (glycerol-3-phosphate | 8.64446321 | 0.000223 | 0.005743 |
| | transporter), member 1?? | | | |
| Ppp4r3b | protein phosphatase 4 regulatory subunit 3B | 1.32854854 | 0.000226 | 0.005816 |
| Zbtb20 | zinc finger and BTB domain containing 20 | 1.12942385 | 0.000118 | 0.003366 |

### An Curly Consequence of Ndnfs2 Deletion was Axonal Dysfunction

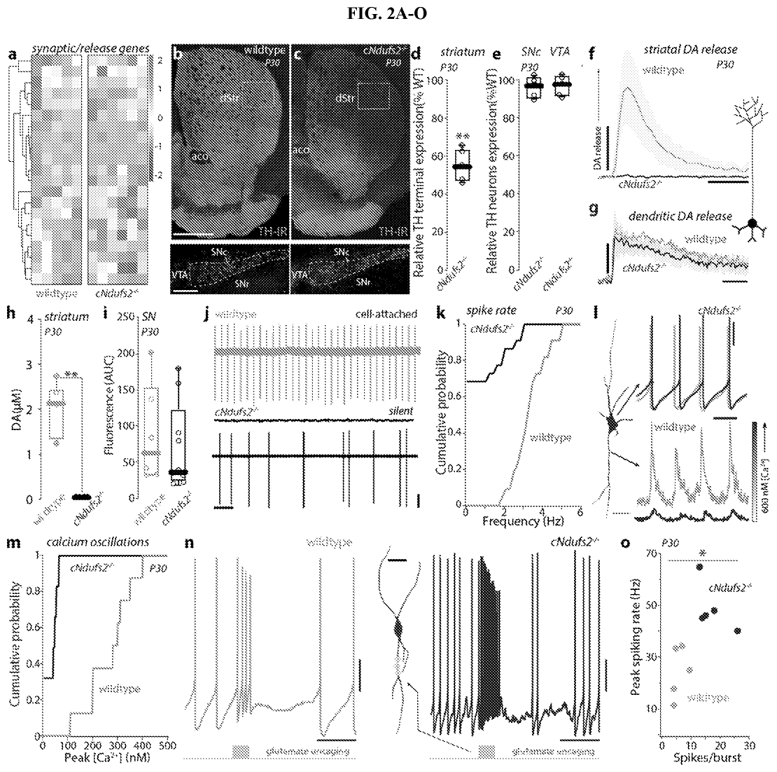

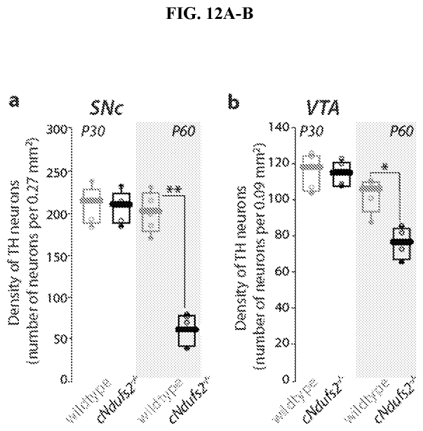

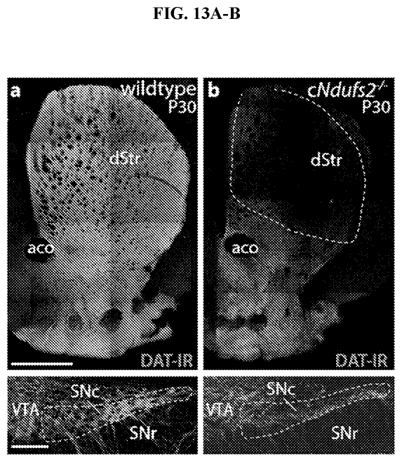

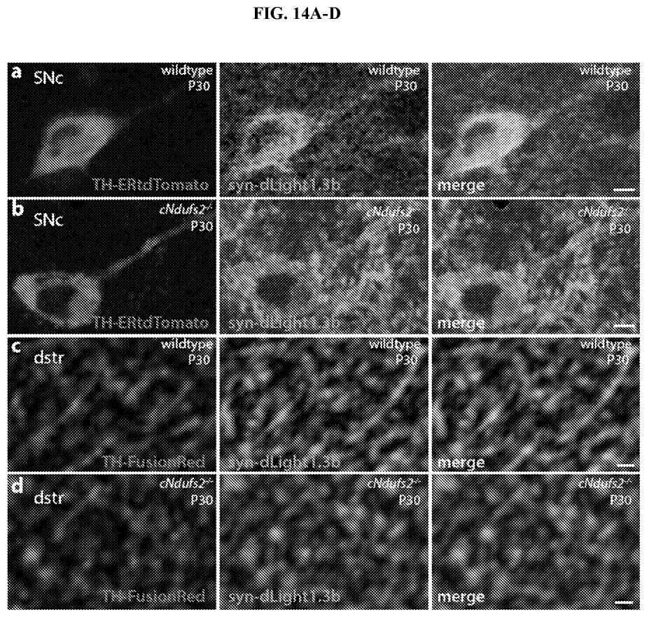

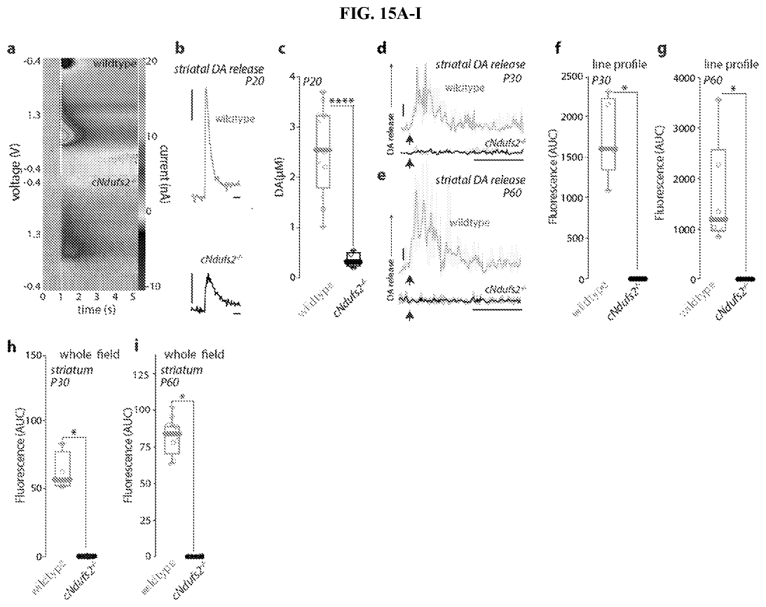

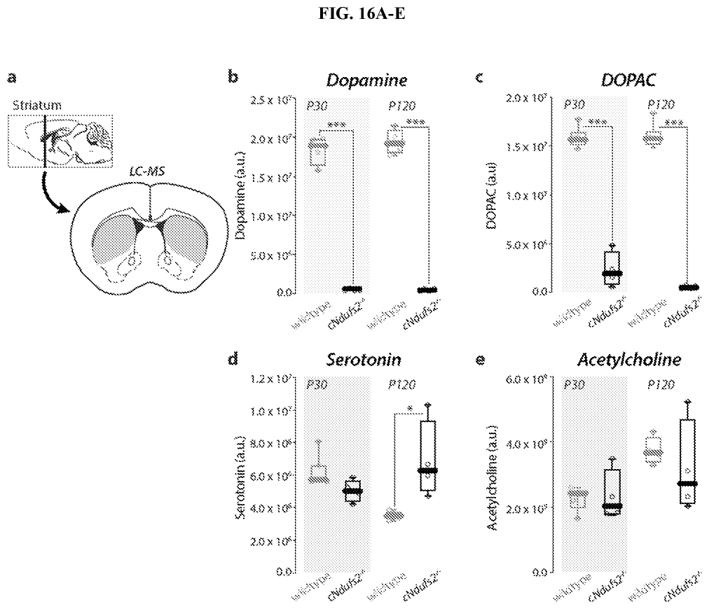

In addition to triggering metabolic reprogramming, loss of Ndufs2 induced significant changes in the expression of genes related to axonal growth and transport (e.g., Tubb3, Uchl1, WntSa, Sema3g Nofl, Nefm, Prkca, Sema4d), synaptic transmission (e.g., Syt1/3/17, Syn2, SCNA), DA synthesis/storage (e.g., TH, VAMP2) and presynaptic regulation (e.g., Drd2, Chra4,6) (FIG. 2a, Tables 4-6). Both tyrosine hydroxylase (TH) (FIG. 2b-d, FIG. 12) and dopamine transporter (DAT) (FIG. 13) protein levels in the basal ganglia were down-regulated in the dorsal striatum by around P30, paralleling the loss of mitochondrial OXPHOS. In contrast, TH expression was much less affected in the ventral striatum (FIG. 2b, c). Moreover, the number of TH-immunoreactive (IR) neurons in the SN and VTA was unchanged at this time point (FIGS. 2b, c and e, FIG. 12). To determine the functional significance of these changes, DA release evoked by electrical stimulation was measured in ex vivo brain slices using fast scan cyclic voltammetry or the genetically-encoded, optical DA sensor dLight (Ref. 19; incorporated by reference in its entirety). By P30, DA release by dopaminergic axons in the dorsolateral striatum-measured with either approach—was close to zero. In contrast, somatodendritic DA release in the SN (measured with dLight) was normal (FIG. 2f-i, FIG. 14-15). The deficit in evoked striatal DA release was validated using liquid chromatography and mass spectrometry analysis of striatal tissue, confirming that there was a profound drop in striatal DA synthesis in cNdufs2^{-/-} mice (FIG. 16).

TABLE 4
Dopamine release - P40

| | | Fold Change | | |
|---|---|---|---|---|
| Gene | Description | (log₂) | p Value | FDR |
| Rab3b | RAB3B, member RAS oncogene family | 1.0720336 | 4.77E−05 | 0.001613 |
| Syn2 | synapsin II | 0.7573761 | 0.001885 | 0.030668 |
| Rims1 | regulating synaptic membrane exocytosis 1 | −0.580048 | 0.002118 | 0.033517 |
| Syt1 | synaptotagmin I | −0.859763 | 1.89E−15 | 5.79E−13 |
| Pcp4 | Purkinje cell protein 4 | −0.584419 | 7.08E−06 | 0.000318 |
| Gabbr1 | gamma-aminobutyric acid (GABA) B receptor, 1 | −0.326781 | 0.001156 | 0.021268 |
| Chrna4 | cholinergic receptor, nicotinic, alpha polypeptide 4 | −1.149198 | 3.16E−34 | 8.71E−31 |
| Slc6a3 | solute carrier family 6 (neurotransmitter transporter, | −1.602839 | 5.00E−57 | 1.73E−53 |
| | dopamine), member 3 | | | |
| Sncg | synuclein, gamma | −1.652030 | 3.30E−10 | 2.27E−10 |
| Cplx1 | complexin 1 | −1.927701 | 4.16E−79 | 1.91E−75 |
| Apba1 | amyloid beta (A4) precursor protein binding, family A, | −0.709018 | 2.15E−07 | 1.40E−05 |
| | member 1 | | | |
| Slc18a2 | solute carrier family 18 (vesicular monoamine), member 2 | −0.773733 | 4.05E−09 | 3.35E−07 |
| Syt17 | synaptotagmin XVII | −0.452197 | 0.003199 | 0.045225 |
| Lin7b | lin-7 homolog B (C. elegans) | −0.795575 | 1.32E−05 | 0.000538 |
| Syt3 | synaptotagmin III | −0.632780 | 0.001036 | 0.019660 |
| Vamp2 | vesicle-associated membrane protein 2 | −0.450227 | 1.69E−07 | 1.12E−05 |
| Park7 | Parkinson disease (autosomal recessive, early onset) 7 | −0.329815 | 0.000275 | 0.006888 |
| Snca | synuclein, alpha | −0.647597 | 3.16E−12 | 3.85E−10 |
| Drd2 | dopamine receptor D2 | −0.990070 | 8.70E−12 | 9.92E−10 |
| Th | tyrosine hydroxylase | −0.472648 | 0.000130 | 0.003653 |
| Chrna6 | cholinergic receptor, nicotinic, alpha polypeptide 6 | −0.991304 | 1.25E−10 | 1.22E−08 |

TABLE 5
Presynaptic proteins - P40

| | | Fold Change | | |
|---|---|---|---|---|
| Gene | Description | (log₂) | p Value | FDR |
| Syt3 | synaptotagmin III | −0.6327805 | 0.001036 | 0.019660 |
| Stx3 | syntaxin 3 | −0.9924268 | 0.000665 | 0.013858 |
| Syt17 | synaptotagmin XVII | −0.4521975 | 0.003199 | 0.045225 |
| Cplx2 | complexin 2 | −0.6340242 | 1.64E−05 | 0.000650 |
| Syngr3 | synaptogyrin 3 | −0.4776772 | 6.40E−05 | 0.002067 |
| Citb | clathrin, light polypeptide (Lcb) | −0.3153029 | 0.000595 | 0.012693 |
| Chrna4 | cholinergic receptor, nicotinic, alpha polypeptide 4 | −1.1491985 | 3.16E−34 | 8.71E−31 |
| Slc6a3 | solute carrier family 6 (neurotransmitter transporter, | −1.6028397 | 5.00E−57 | 1.73E−53 |
| | dopamine), member 3 | | | |
| Sncg | synuclein, gamma | −1.6520305 | 3.30E−10 | 2.27E−10 |
| Cplx1 | complexin 1 | −1.9277017 | 4.16E−79 | 1.91E−75 |
| Rab3c | RAB3C, member RAS oncogene family | −1.1362676 | 1.88E−20 | 1.13E−17 |
| Syt1 | synaptotagmin I | −0.8597631 | 1.89E−15 | 5.79E−13 |
| Sv2c | synaptic vesicle glycoprotein 2c | −1.3462421 | 1.14E−29 | 1.96E−26 |
| Ap2s1 | adaptor-related protein complex 2, sigma 1 subunit | −0.5319392 | 9.05E−06 | 0.000389 |
| Sncb | synuclein, beta | −0.5102844 | 2.83E−05 | 0.001047 |
| Slc18a2 | solute carrier family 18 (vesicular monoamine), member 2 | −0.7737334 | 4.05E−09 | 3.35E−07 |
| Vsnl1 | visinin-like 1 | −0.6988869 | 2.59E−07 | 1.65E−05 |
| Ptpn5 | protein tyrosine phosphatase, non-receptor type 5 | −1.6015279 | 2.80E−26 | 3.22E−23 |
| Snca | synuclein, alpha | −0.6475971 | 3.16E−12 | 3.85E−10 |
| Drd2 | dopamine receptor D2 | −0.9900701 | 8.70E−12 | 9.92E−10 |
| Chrna6 | cholinergic receptor, nicotinic, alpha polypeptide 6 | −0.9913042 | 1.25E−10 | 1.22E−08 |
| Chrna3 | cholinergic receptor, nicotinic, alpha polypeptide 3 | −4.0555417 | 0.000448 | 0.010207 |

TABLE 6
Dendritic transmitter release - P40

| | | Fold Change | | |
|---|---|---|---|---|
| Gene | Description | (log₂) | p Value | FDR |
| Syn2 | synapsin II | 0.75737612 | 0.001880 | 0.030668 |
| Gcc2 | GRIP and coiled-coil domain containing 2 | 0.72707164 | 0.000174 | 0.004656 |
| Cplx2 | complexin 2 | −0.6340242 | 1.64E−05 | 0.000650 |
| Rims1 | regulating synaptic membrane exocytosis 1 | −0.5800481 | 0.002118 | 0.033517 |
| Syt17 | synaptotagmin XVII | −0.4521975 | 0.003199 | 0.045225 |
| Grm1 | glutamate receptor, metabotropic 1 | −1.6281874 | 3.80E−05 | 0.001327 |
| Vamp2 | vesicle-associated membrane protein 2 | −0.450227 | 1.69E−07 | 1.12E−05 |
| Stx3 | syntaxin 3 | −0.9924268 | 0.000665 | 0.013858 |
| Cpne6 | copine VI | −1.1615958 | 2.33E−09 | 2.01E−07 |
| Syt3 | synaptotagmin III | −0.6327805 | 0.001036 | 0.019660 |
| Gap43 | growth associated protein 43 | −0.4711051 | 4.61E−07 | 2.76E−05 |
| Syt1 | synaptotagmin I | −0.8597631 | 1.89E−15 | 5.79E−13 |
| Cplx1 | complexin 1 | −1.9277017 | 4.16E−79 | 1.91E−75 |
| Snapc5 | small nuclear RNA activating complex, polypeptide 5 | −0.8978219 | 1.90E−05 | 0.000747 |
| Homer2 | homer scaffolding protein 2 | −0.8899711 | 6.04E−05 | 0.001965 |

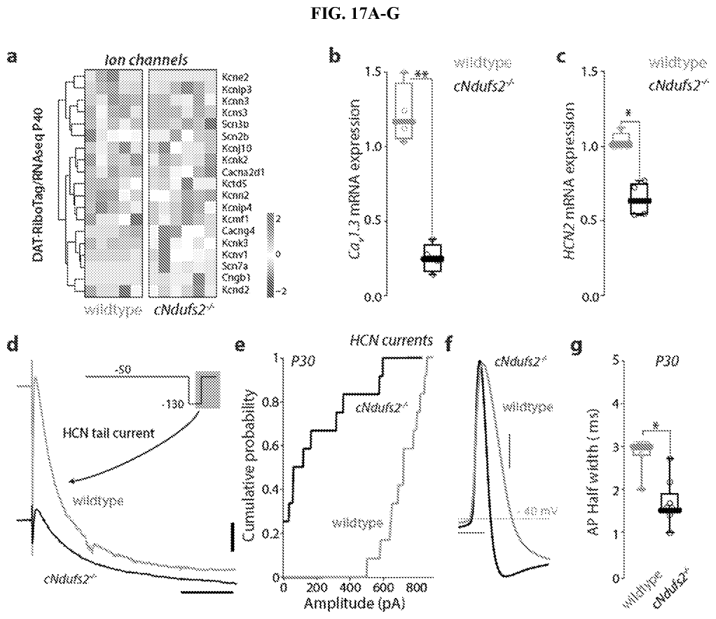

Although somatodendritic release of DA by SN dopaminergic neurons was not discernibly altered by Ndufs2 deletion at this point in time, the physiology of this sub-cellular region was changed. In wildtype SN dopaminergic neurons, this cellular region is invested with ion channels that drive slow, autonomous pacemaking with broad spikes (Refs. 20, 21; incorporated by reference in their entireties). But in cell-attached recordings from P30-40 SN dopaminergic neurons from cNdufs2^{-/-} mice, pacemaking had slowed or stopped (FIG. 2j, k). The translatomes of these cells revealed an up-regulation in K+ channels (e.g., TASK-1, TREK-1 channels) that might suppress pacemaking (FIG. 17a, Table 7). In addition, expression of Mink-related peptide 1 (Kcne2), which positively regulates hyperpolarization-activated cyclic nucleotide-gated (HCN) cation channel function (Ref. 22; incorporated by reference in its entirety) was downregulated, as was expression of HCN2 and HCN3 subunits (FIG. 17c, Table 7). Indeed, HCN channel currents, which help drive pacemaking, were reduced in cNdufs2-SN dopaminergic neurons (FIG. 17d, e). Another important pacemaking-related current flows through Cav1.3 channels and is responsible for large cytosolic Ca2+transients that drive mitochondrial OXPHOS7. In cNdufs2^{-/-} SN dopaminergic neurons, expression of mRNA for the pore-forming subunit of Cav1.3 channels was down-regulated and cytosolic Ca2+transients evoked by spiking were nearly abolished (FIG. 2l, m, FIG. 17b, Table 7). In contrast, the expression of mRNAs coding for glutamate receptors that drive burst spiking appeared to be unchanged in cNdufs2^{-/-} neurons (Table 8). Indeed, the dendritic arbors of ˜P40 cNdufs2^{-/-} SN dopaminergic neurons were normal in appearance (FIGS. 2l and n) and two photon uncaging of glutamate along them evoked bursts of spikes. These bursts were higher in frequency and longer in duration than those evoked in wildtype neurons, reflecting in part the dramatic shortening of spike duration (FIGS. 2n-o. FIG. 17f, g).

TABLE 7
Ion channels - P40

| | | Fold Change | | |
|---|---|---|---|---|
| Gene | Description | (log₂) | p Value | FDR |
| Kcne2 | potassium voltage-gated channel, Isk-related subfamily, | −23.341683 | 4.40E−16 | 1.44E−13 |
| | gene 2 | | | |
| Kcnip3 | Kv channel interacting protein 3, calsenilin | −0.9573277 | 0.000874 | 0.017424 |
| Kcnn3 | potassium intermediate/small conductance calcium- | −0.8702095 | 0.001337 | 0.023649 |
| | activated channel, subfamily N, member 3 | | | |
| Kcns3 | potassium voltage-gated channel, delayed-rectifier, | −1.8624933 | 9.27E−15 | 2.37E−12 |
| | subfamily S, member 3 | | | |
| Scn3b | sodium channel, voltage-gated, type III, beta | −0.6340787 | 0.000316 | 0.007680 |
| Scn2b | sodium channel, voltage-gated, type II, beta | −0.5989172 | 0.002036 | 0.032396 |
| Kcnj10 | potassium inwardly-rectifying channel, subfamily J, | 0.84367987 | 0.000975 | 0.018785 |
| | member 10 | | | |
| Kcnk2 | potassium channel, subfamily K, member 2 | 1.01612359 | 0.000984 | 0.018914 |
| Cacna2d1 | calcium channel, voltage-dependent, alpha2/delta subunit 1 | 1.00667539 | 9.59E−05 | 0.002850 |
| Kctd5 | potassium channel tetramerisation domain containing 5 | 1.09819336 | 0.002921 | 0.042148 |
| Kcnn2 | potassium intermediate/small conductance calcium- | 1.17800857 | 0.001512 | 0.026187 |
| | activated channel, subfamily N, member 2 | | | |
| Kcnip4 | Kv channel interacting protein 4 | 0.68643640 | 4.56E−05 | 0.001554 |
| Kcmf1 | potassium channel modulatory factor 1 | 0.69188177 | 0.001810 | 0.029799 |
| Cacng4 | calcium channel, voltage-dependent, gamma subunit 4 | 1.04541122 | 0.000513 | 0.011276 |
| Kcnk3 | potassium channel, subfamily K, member 3 | 2.50660353 | 5.73E−05 | 0.001891 |
| Kcnv1 | potassium channel, subfamily V, member 1 | 5.23304007 | 0.001819 | 0.029859 |
| Scn7a | sodium channel, voltage-gated, type VII, alpha | 8.31992375 | 0.001704 | 0.028530 |
| Cngb1 | cyclic nucleotide gated channel beta 1 | 6.26788288 | 0.002113 | 0.033472 |
| Kcnd2 | potassium voltage-gated channel, Shal-related family | 1.12995839 | 0.002313 | 0.035700 |
| | member 2 | | | |
| Cacna1d(*) | calcium channel, voltage-dependent, L type, alpha 1D | −0.0378691 | 0.953790 | 0.993263 |
| | subunit | | | |
| Hcn2(*) | hyperpolarization-activated, cyclic nucleotide-gated K + 2 | −0.3518609 | 0.1179361 | 0.473460 |
| Hcn3 | hyperpolarization-activated, cyclic nucleotide-gated K + 3 | −0.5922747 | 0.0804090 | 0.384858 |

*qPCR Validation

TABLE 8
Glutamate receptors - P40

| | | Fold Change | | |
|---|---|---|---|---|
| Gene | Description | (log₂) | p Value | FDR |
| Grik5 | glutamate receptor, ionotropic, kainate 5 (gamma 2) | −0.59250 | 0.00757 | 0.08328 |
| Grin2a | glutamate receptor, ionotropic, NMDA2A (epsilon 1) | 1.06996 | 0.00948 | 0.09783 |
| Gria4 | glutamate receptor, ionotropic, AMPA4 (alpha 4) | −0.48190 | 0.01644 | 0.14031 |
| Gria3 | glutamate receptor, ionotropic, AMPA3 (alpha 3) | 0.607027 | 0.04569 | 0.27527 |
| Grina | glutamate receptor, ionotropic, N-methyl D-aspartate- | 0.162131 | 0.08294 | 0.39073 |
| | associated protein 1 (glutamate binding) | | | |
| Grin2c | glutamate receptor, ionotropic, NMDA2C (epsilon 3) | −2.52990 | 0.10936 | 0.45500 |
| Gria2 | glutamate receptor, ionotropic, AMPA2 (alpha 2) | 0.256230 | 0.14151 | 0.51616 |
| Grik1 | glutamate receptor, ionotropic, kainate 1 | 1.010912 | 0.16166 | 0.55062 |
| Grin2d | glutamate receptor, ionotropic, NMDA2D (epsilon 4) | 0.345867 | 0.35352 | 0.77653 |
| Gria1 | glutamate receptor, ionotropic, AMPA1 (alpha 1) | 0.193471 | 0.35366 | 0.77657 |
| Grid2 | glutamate receptor, ionotropic, delta 2 | 1.066328 | 0.42126 | 0.82777 |
| Grik3 | glutamate receptor, ionotropic, kainate 3 | 0.145830 | 0.65339 | 0.93602 |
| Grin2b | glutamate receptor, ionotropic, NMDA2B (epsilon 2) | −0.13657 | 0.67679 | 0.93943 |
| Grin1 | glutamate receptor, ionotropic, NMDA1 (zeta 1) | −0.09887 | 0.72915 | 0.95135 |
| Grik2 | glutamate receptor, ionotropic, kainate 2 (beta 2) | −0.19197 | 0.83834 | 0.97543 |
| Grid1 | glutamate receptor, ionotropic, delta 1 | 0.075420 | 0.86778 | 0.98016 |
| Grik4 | glutamate receptor, ionotropic, kainate 4 | 0.041213 | 0.94382 | 0.99223 |
| Grid2ip | glutamate receptor, ionotropic, delta 2 (Grid2) | 1.023818 | 0.63918 | NA |
| | interacting protein 1 | | | |
| Grin3b | glutamate receptor, ionotropic, NMDA3B | NA | NA | NA |
| Grin1os | glutamate receptor, ionotropic, NMDA1 (zeta 1), | NA | NA | NA |
| | opposite strand | | | |
| Grm1 | glutamate receptor, metabotropic 1 | −1.62818 | 3.8E−05 | 0.00132 |
| Grm8 | glutamate receptor, metabotropic 8 | 2.242760 | 0.08919 | 0.40984 |
| Grm7 | glutamate receptor, metabotropic 7 | 1.243327 | 0.15962 | 0.54731 |
| Grm3 | glutamate receptor, metabotropic 3 | 0.541041 | 0.55077 | 0.89407 |
| Grm5 | glutamate receptor, metabotropic 5 | −0.14408 | 0.56693 | 0.90253 |
| Grm4 | glutamate receptor, metabotropic 4 | −0.30114 | 0.64839 | 0.93348 |
| Grm2 | glutamate receptor, metabotropic 2 | 0.278566 | 0.68870 | 0.94190 |
| Grm6 | glutamate receptor, metabotropic 6 | NA | NA | NA |

### Progressive Loss of Somatodendritic Phenotype Followed Axonal Dysfunction

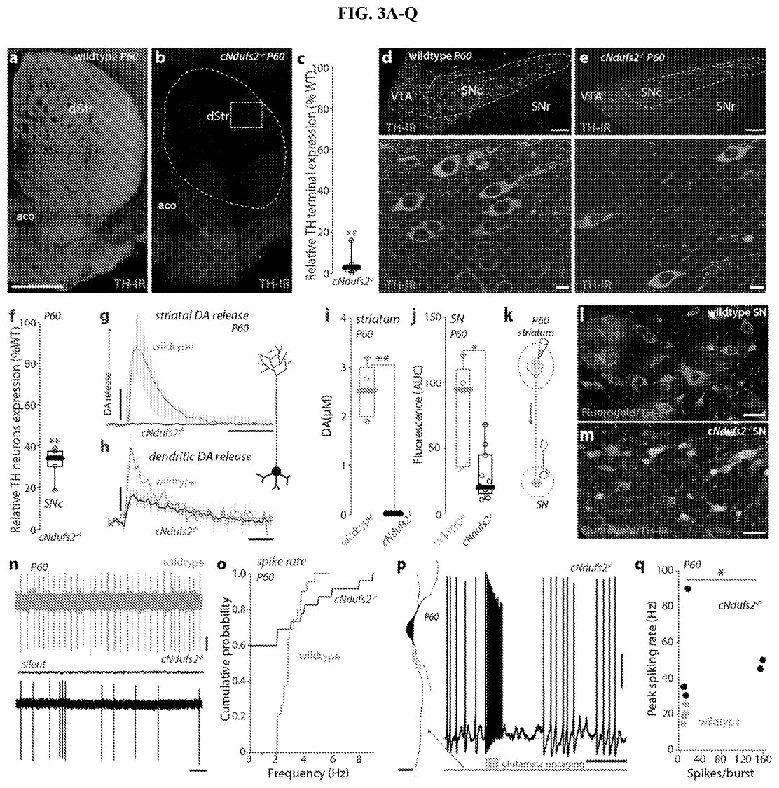

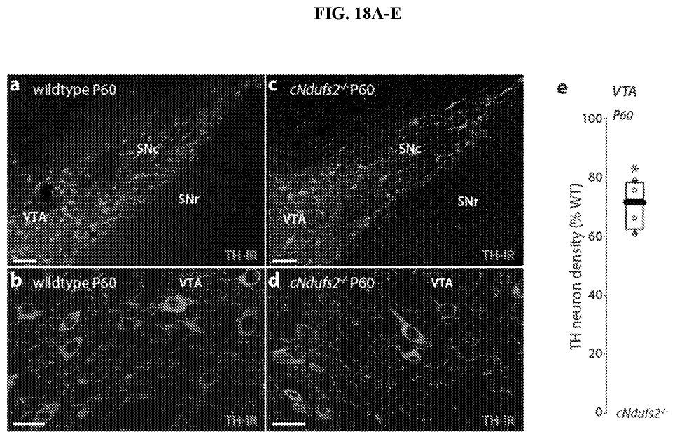

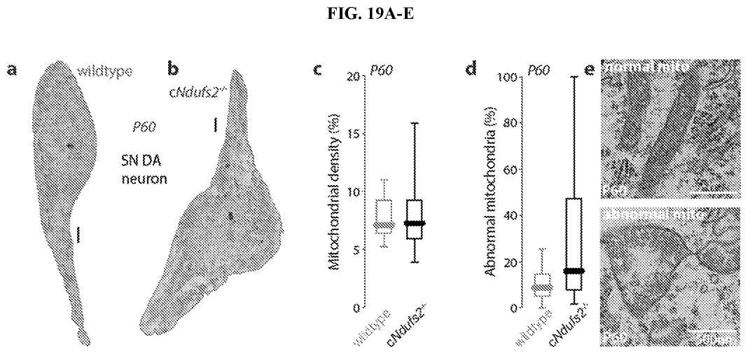

By P60, the loss of axonal proteins associated with dopaminergic signaling expanded to include the ventral striatum (FIGS. 3a-c, FIG. 12). In addition, TH expression in the somatodendritic region of cNdufs24-SN dopaminergic neurons fell to roughly half that of age-matched controls (FIGS. 3d-f, FIG. 12). TH expression also fell in cNdufs2^{-/-} VTA dopaminergic neurons (FIG. 12, FIG. 18). In parallel, the release of DA in the SN fell by roughly 75% (FIGS. 3h, j). To determine if these functional changes were attributable to degeneration of dopaminergic axons and cell bodies, the retrogradely transported marker Fluoro-Gold was injected into the striatum of P60 (+4 days) cNdufs2^{-/-} mice; five days later animals were sacrificed for histological analysis. Many cNdufs2^{-/-} SN neurons were labeled; in fact, the number of retrogradely labeled neurons were similar in cNdufs2^{-/-} and wildtype mice (wildtype cells=235, IQR: 303; cNdufs2^{-/-} cells=207, IQR: 317.5; N=4 per group, FIG. 3k-m). Thus, at this stage in the evolution of pathology in the cNdufs2^{-/-} mice, the loss of markers for dopaminergic signaling reflected a phenotypic down-regulation, rather than frank neurodegeneration. Moreover, mitochondrial density in cNdufs2^{-/-} dopaminergic neurons was unchanged at this time point (FIG. 19).

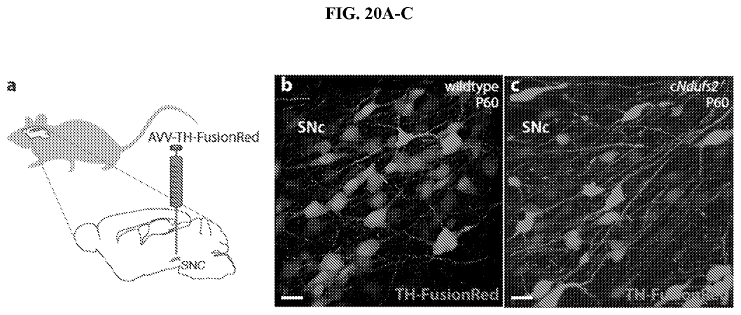

To determine if the changes in somatodendritic TH expression and DA release were matched by alterations in electrophysiology, dopaminergic neurons were labeled by injecting an adenoassociated virus (AAV) into the SN of P30 mice that carried a TH promoter driven FusionRed (FR) reporter construct. At P60 (+4 days), FR-labeled neurons in ex vivo brain slices from cNdufs2″ mice were studied (FIG. 20). As at P30, many cNdufs2^{-/-} SN dopaminergic neurons were silent and in those that did spike did so irregularly (FIGS. 3n, o). Nevertheless, as at P30, the dendritic arbor of cNdufs2^{-/-} SN dopaminergic neurons appeared to be intact and dendritic uncaging of glutamate evoked a robust, high frequency burst of somatic spikes (FIG. 3p, q). Thus, the decline in somatodendritic transmitter phenotype of cNdufs2^{-/-} SN dopaminergic neurons was not closely matched by a loss in responsiveness to extrinsic excitatory stimuli.

cNdufs2^{-/-} Mice Manifested a Progressive, Levodopa-Responsive Parkinsonism

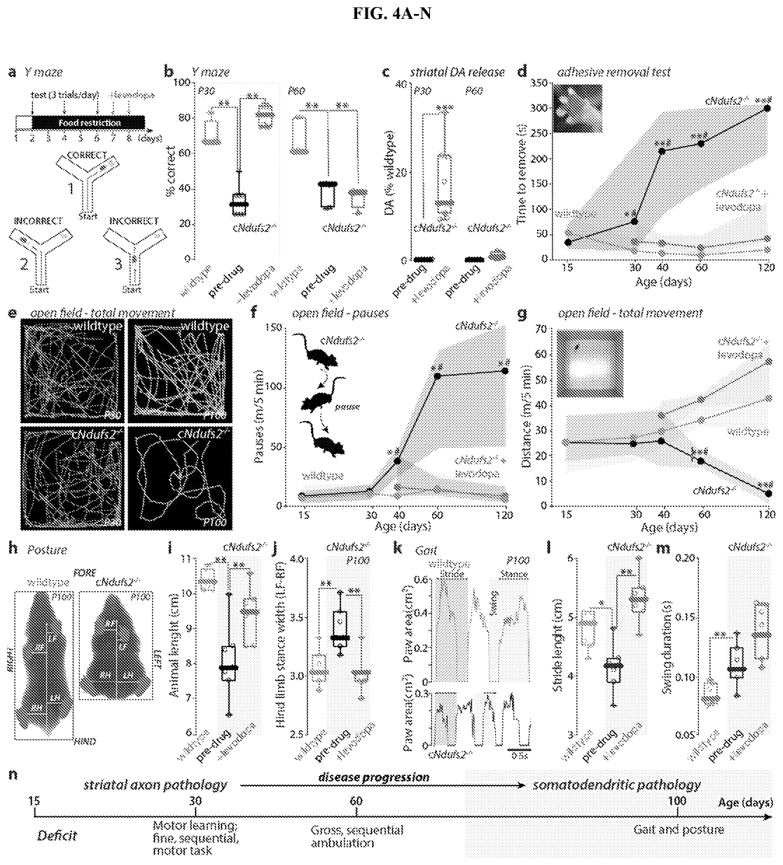

Unlike conventional PD models in which DA is depleted rapidly throughout the basal ganglia, the staging of pathology in cNdufs2^{-/-} mice allowed an assessment of how regional deficits in DA release are coupled to behavior. Again, through weaning, cNdufs2^{-/-} mice were indistinguishable from littermate controls. However, as dorsal striatal DA release declined to near detection thresholds around P30, cNdufs2^{-/-} mice lost the ability to perform an associative learning task that is thought to rely upon DA-dependent striatal synaptic plasticity (FIGS. 4a, b)23. This memory task was restored by systemic levodopa treatment (6 mg/kg) at P30, but not by treatment later at P60 (FIG. 4b). As associative synaptic plasticity in this task is widely thought to depend upon appropriately timed, phasic DA release (Ref. 23; incorporated by reference in its entirety), the ability of levodopa treatment to restore evoked striatal DA release was determined in ex vivo brain slices from mice at these two ages. In striatal tissue from P30 mice, but not from P60 mice, levodopa treatment was able to restore evoked DA release (FIG. 4c).

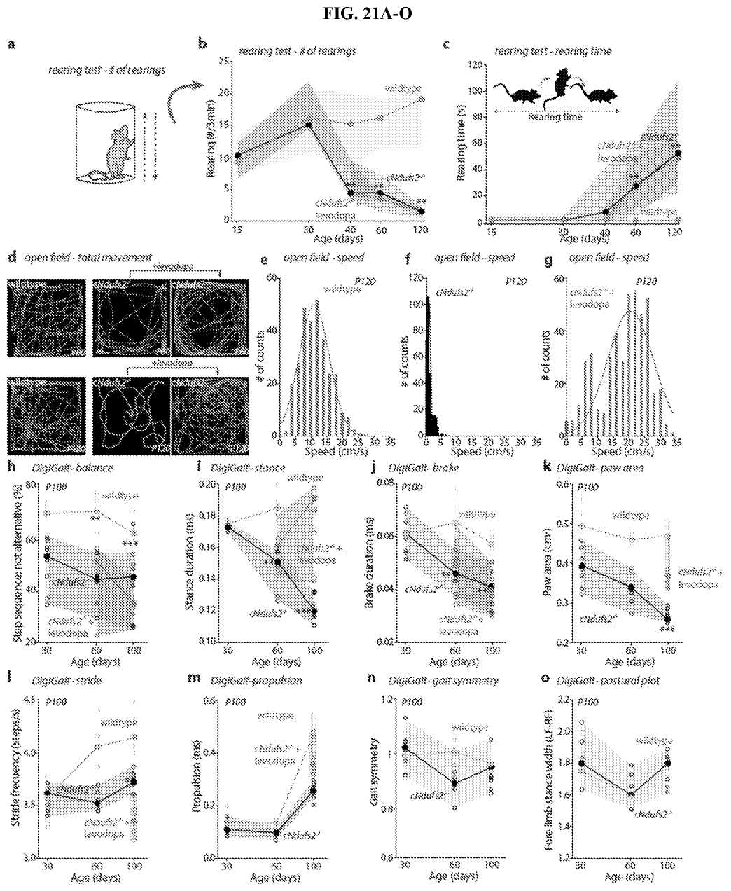

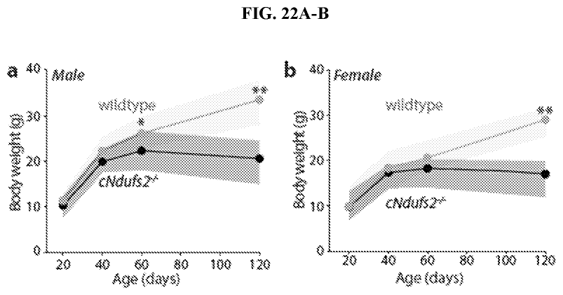

Although deficits in striatal motor learning were profound at P30, mice did not exhibit gross impairments in motor performance. That said, fine motor skill, as assessed by the time taken to remove an adhesive from the forepaw, was significantly slowed in cNdufs2^{-/-} mice at this age and became progressively worse with time (FIG. 4d). Unlike the situation with the associative learning task, levodopa treatment improved adhesive removal speed regardless of age (FIG. 4d). By P40, open-field, exploratory behavior of cNdufs2″ mice began to be interrupted by brief pauses, although the total distance traveled in the testing period was normal (FIGS. 4e-g). Later, open-field behavior of cNdufs2^{-/-} mice became progressively more impaired and total distance traveled began to fall (FIG. 4g). As expected of a disability rooted in dopaminergic signaling, levodopa treatment improved performance in this task (FIGS. 4f, g, FIGS. 21d-g). Another common exploratory behavior of mice involves rearing. When placed in a tall glass cylinder, mice rear, land and rear again in a different part of the cylinder; in a 3-minute period, mice typically rear and land 15-20 times. At P30, rearing behavior of cNdufs2^{-/-} mice was normal, but by P60 cNdufs2^{-/-} mice appeared to have difficulty transitioning between rearing and landing, spending much more time ‘stuck’ in an elevated posture. Levodopa treatment did not lessen this deficit (FIGS. 21a-c). After the appearance of these gross motor deficits, the weight of cNdufs2^{-/-} mice stabilized and diverged from that wildtype mice, possibly because of difficulty feeding (FIG. 22).

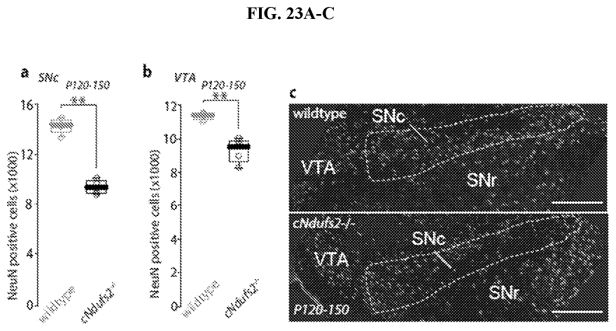

Despite the slowing of movement in the open field, cNdufs2^{-/-} mice at around P60 exhibited only subtle impairments in gait when placed on a treadmill (FIGS. 21h-o). However, by P100, cNdufs2^{-/-} mice had splayed hindlimbs, abnormal paw placement and alterations in stride when forced to ambulate on the treadmill (FIG. 4h-m, FIG. 21h-o). Levodopa ameliorated some, but not all of these deficits (FIG. 4i-m, FIG. 21h-o). In this timeframe (P120-150), roughly 40% of SN dopaminergic neurons appear to have been lost, suggesting that phenotypic down-regulation preceded frank cell death (FIG. 23). Because cNdufs2^{-/-} mice manifest a clear, levodopa-responsive Parkinsonism that is attributable to MCI dysfunction, they are referred to as MCI-Park mice.

### Boosting Mesencephalic Dopaminergic Signaling Alleviated Motor Disability

The late emergence of gross motor deficits in MCI-Park mice, which paralleled the changes in SN DA release rather than release in the dorsal striatum, poses a conceptual problem for the theory of network dysfunction underlying PD motor symptoms that has been in place for over 30 years (Ref. 24; incorporated by reference in its entirety). This network theory posits that the imbalance between the activity of direct and indirect striatal efferent pathways created by striatal DA depletion is the prime driver of clinical PD symptoms (bradykinesia, rigidity). While there is unequivocal clinical evidence that striatal DA depletion is necessary for bradykinesia and rigidity in PD patients (Ref. 25; incorporated by reference in its entirety), the sufficiency of striatal pathophysiology has never been adequately tested because commonly used models induce rapid DA depletion throughout the basal ganglia. The staging of DA depletion in MCI-Park mice paints a different picture. This model indicates that although the loss of dorsal striatal DA release is sufficient to produce motor learning and fine movement deficits, it is not sufficient to bring about a state resembling clinical PD. This conclusion is consistent with previous work showing the benefit SN dopaminergic neuron transplants in PD models (Refs. 26, 27; incorporated by reference in its entirety), as well as examination of how PD models respond to pharmacological manipulation of the SN (Ref. 28; incorporated by reference in its entirety).

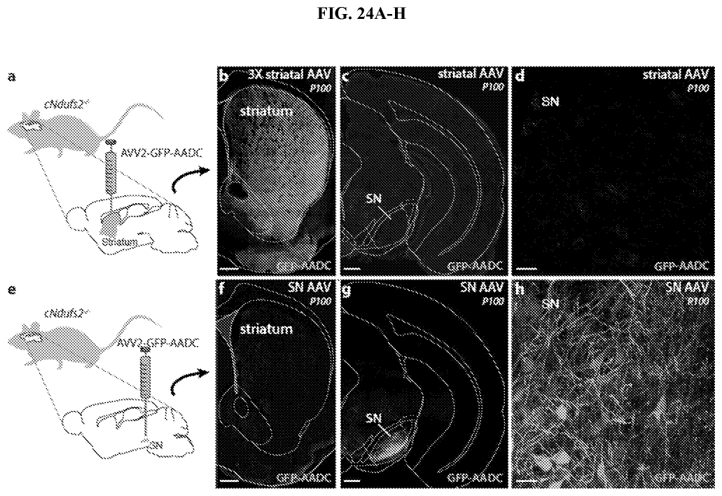

Despite these observations and the recognition that DA modulates synaptic function throughout the basal ganglia, the striatocentric model of PD remains firmly entrenched and shapes treatment of PD patients (Ref. 25; incorporated by reference in its entirety). For example, in late-stage PD patients, the efficacy of levodopa begins to wane as brain levels of aromatic acid decarboxylase (AADC), which convert levodopa to DA, fall. To overcome this deficit, an AADC gene therapy is being tested that targets exclusively the striatum (NCT01973543). In agreement with previous work (Ref. 29; incorporated by reference in its entirety), stereotaxic injection of an AAV carrying an AADC-GFP expression construct into the striatum (FIGS. 5a-d, FIGS. 24a-d) of MCI-Park mice enhanced ambulation in response to a low (1.5 mg/kg) systemic dose of levodopa, which in control MCI-Park mice had no discernible effect (FIGS. 5m and o). As DA does not cross the blood-brain barrier and has a limited ability to diffuse in the brain parenchyma (Ref. 30; incorporated by reference in its entirety), this effect is consistent with the proposition that striatal DA depletion is necessary for the ambulatory impairment in MCI-Park mice and in PD patients.

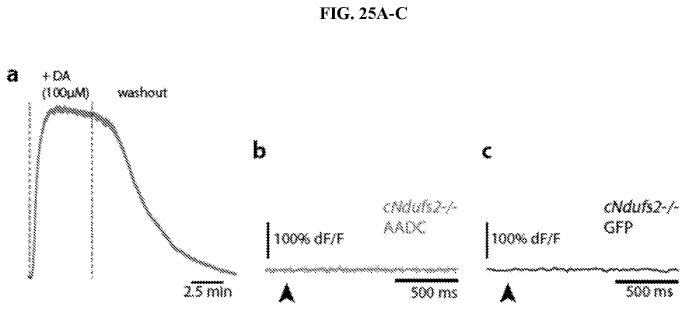

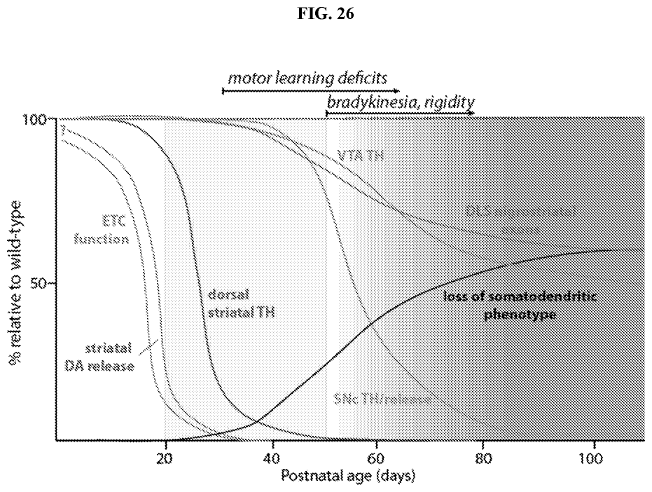

Experiments were conducted during development of embodiments herein to determine whether striatal DA depletion sufficient to cause the ambulation deficit. AAV-AADC was stereotaxically injected into the SN and the same low dose of levodopa given at P100. (FIGS. 5e-h. FIGS. 24e-h). As predicted by the behavioral staging, SN expression of AADC was just as effective in boosting the impact of a low dose of levodopa on open-field ambulation as was striatal expression of AADC (FIGS. 5n, o). This effect was not attributable to elevation of striatal conversion of levodopa to dopamine, as SN AADC expression did not increase evoked striatal dopamine release or dopamine metabolites following levodopa treatment (FIGS. 5i-1, FIG. 25). These results are consistent with the conclusion that both striatal and SN DA depletion are necessary for the emergence of PD-like deficits in ambulation (FIG. 5p, FIG. 26).

### DISCUSSION

Loss of MCI function in dopaminergic neurons is sufficient to trigger a progressive, axon-first loss of function and levodopa-responsive Parkinsonism. DA depletion in the dorsal striatum is necessary, but not sufficient to produce the gross movement deficits associated with clinical Parkinsonism. Rather, the emergence of this level of impairment required a loss of SN DA release as well.

Although long known to be correlated with PD, the consequences of acquired deficits in MCI function have been unclear. Unlike the situation created by systemic administrations of toxins, like 1-methyl-4-phenyl-1,2,3,6-tetrahydropyridine, the slow loss of MCI function induced by Ndufs2 deletion allowed dopaminergic neurons in the brain the time necessary to reprogram metabolism and physiology—as would be the case in a slowly evolving disease, like human PD. In this context, the resilience of SN dopaminergic neurons to partial loss of MCI function is understandable Ref. 8, 9; incorporated by reference in their entireties).

But the switch from a reliance upon mitochondrial OXPHOS to glycolysis was not without consequences. One of the distinguishing features of SN dopaminergic neurons and other neurons at risk in PD is their massive striatal axonal arbor (Ref. 31; incorporated by reference in its entirety). In contrast to the somatodendritic region, this arbor's high surface area to volume ratio, unique ionic environment and density of transmitter release sites appears to create a local environment in which the metabolic efficiency of mitochondrial OXPHOS is indispensable (Refs. 6, 32; incorporated by reference in their entireties). Indeed, disrupting axonal transport of mitochondria in dopaminergic neurons by deleting Drp1 triggered a similar SN-specific loss of function, leaving neighboring VTA dopaminergic neurons, which have less branched axons, significantly less affected as seen here (Ref. 33; incorporated by reference in its entirety). These results provide a framework for understanding how declining mitochondrial OXPHOS capacity with aging could result in the early loss of nigrostriatal axons characteristic of idiopathic PD34.

The loss of mitochondrial OXPHOS also led to a seismic shift in somatodendritic properties. The Warburg-like transformation of cNdufs2^{-/-} dopaminergic neurons was paralleled by spike narrowing, suppression of spike-evoked cytosolic Ca2+transients and inhibition of autonomous spike generation. A simple take-away from these events is that the primary goal of this set of physiological traits is to drive mitochondrial OXPHOS Refs. 5.7; incorporated by reference in their entireties). When this capacity was lost, the cell shed this phenotype. With time, dopaminergic traits were down-regulated in this region as well.

Unlike most models of PD, the progressive staging of pathology in the MCI-Park mouse allowed the behavioral impact of regional DA depletion to be cleanly assessed. The prevailing hypothesis has been that striatal DA depletion produces an imbalance in the excitability of direct and indirect striatal efferent pathways, resulting in disinhibition of basal ganglia output nuclei, suppression of motor control circuits and the defining rigidity and bradykinesia of clinical PD24. While there is ample support for the proposition that striatal DA depletion creates a pathway imbalance Refs. 39, 40; incorporated by reference in their entireties), whether it is sufficient to cause symptoms has not been rigorously tested (Ref. 25; incorporated by reference in its entirety). In the MCI-Park mouse, selective striatal DA depletion produced deficits in associative motor learning and fine sequential motor tasks—both of which have striatal determinants (Refs. 23.41; incorporated by reference in their entireties). Both behavioral deficits may prove to be biomarkers of early-stage PD. But striatal DA depletion alone did not cause the gross motor impairment characteristic of PD. Levodopa-responsive Parkinsonism only appeared when somatodendritic SN DA release fell. The functional role in the basal ganglia of this unusual feature of SN dopaminergic neurons—somatodendritic DA release—has been obscure. The dendrites of SN dopaminergic neurons stretch into the neighboring substantia nigra pars reticulata (SNr), forming an intricate anatomical scaffolding that allows released DA to modulate synaptic terminals arising from the globus pallidus externa (GPe), subthalamic nucleus (STN) and direct pathway SPNs (dSPNs) (Refs. 43.44; incorporated by reference in their entireties). This presynaptic modulation inhibits indirect pathway control of SNr output neurons, while enhancing that of the direct pathway (refs. 45.46; incorporated by reference in their entireties). The staging of motor deficits in MCI-Park mice and the impact of regional AADC expression on the response to systemic levodopa indicate that in the prodromal stages of PD dendritic DA release rebalances direct and indirect pathway control of SNr output created by striatal depletion. In so doing, it may prevent synchronous, rhythmic bursting in GPe and STN from driving a ‘toxic’ patterning of SNr activity and allow telencephalic and brainstem motor circuits to compensate. However, when this rebalancing capacity is lost and toxic patterning of SNr activity emerges, these synaptically coupled motor circuits may themselves become disrupted, resulting in the ambulatory and gait features of PD (Refs 25.45; incorporated by reference in their entireties). In humans, where the internal segment of the globus pallidus is more prominent than in rodents (Ref. 47; incorporated by reference in its entirety), dopamine release from proximal axons may serve a similar role.

### REFERENCES

The following references, some of which are cited above by number, are herein incorporated by reference in their entireties. All references herein are incorporated by reference in their entireties.
1. Poewe, W. et al. Parkinson disease. Nat. Rev. Dis. Primers 3, 17013 (2017).
2. Beilina, A. & Cookson, M. R. Genes associated with Parkinson's disease: regulation of autophagy and beyond. J. Neurochem. 139 Suppl 1, 91-107 (2016).
3. Haelterman, N. A. et al. A mitocentric view of Parkinson's disease. Annu. Rev. Neurosci. 37, 137-159 (2014).
4. Faustini, G. et al. Mitochondria and α-Synuclein: Friends or Foes in the Pathogenesis of Parkinson's Disease? Genes (Basel) 8, 377 (2017).
5. Surmeier, D. J., Obeso, J. A. & Halliday, G. M. Selective neuronal vulnerability in Parkinson disease. Nat. Rev. Neurosci. 18, 101-113 (2017).
6. Bolam, J. P. & Pissadaki, E. K. Living on the edge with too many mouths to feed: why dopaminem neurons die. Mov. Disord. 27, 1478-1483 (2012).
7. Guzman, J. N. et al. Systemic isradipine treatment diminishes calcium-dependent mitochondrial oxidant stress. J. Clin. Invest. 128, 2266-2280 (2018).
8 Kim, H. W. et al. Genetic reduction of mitochondrial complex I function does not lead to loss of dopamine neurons in vivo. Neurobiol. Aging 36, 2617-2627 (2015).
9 Fernández-Agüera, M. C. et al. Oxygen Sensing by Arterial Chemoreceptors Depends on Mitochondrial Complex I Signaling. Cell Metab. 22, 825-837 (2015).
10. Kordower, J. H. & Burke, R. E. Disease Modification for Parkinson's Disease: Axonal Regeneration and Trophic Factors. Mov. Disord. 33, 678-683 (2018).
11. Engblom, D. et al. Glutamate receptors on dopamine neurons control the persistence of cocaine seeking. Neuron 59, 497-508 (2008).
12. Arias-Mayenco, I. et al. Acute O(2) Sensing: Role of Coenzyme QH(2)/Q Ratio and Mitochondrial ROS Compartmentalization. Cell Metab. 28, 145-158.e144 (2018).
13. Sanz, E. et al. Cell-type-specific isolation of ribosome-associated mRNA from complex tissues. Proc. Natl. Acad. Sci. USA 106, 13939-13944 (2009).
14. Fornasiero, E. F. et al. Precisely measured protein lifetimes in the mouse brain reveal differences across tissues and subcellular fractions. Nat. Commun. 9, 4230 (2018).
15. Narendra, D., Walker, J. E. & Youle, R. Mitochondrial quality control mediated by PINK1 and Parkin: links to parkinsonism. Cold Spring Harb. Perspect. Biol. 4 (2012).
16. Baker, N., Patel, J. & Khacho, M. Linking mitochondrial dynamics, cristae remodeling and supercomplex formation: How mitochondrial structure can regulate bioenergetics. Mitochondrion 49, 259-268 (2019).
17. Ekstrand, M. I. et al. Progressive parkinsonism in mice with respiratory-chain-deficient dopamine neurons. Proc. Natl. Acad. Sci. USA 104, 1325-1330(2007).
18. Tantama, M. et al. Imaging energy status in live cells with a fluorescent biosensor of the intracellular ATP-to-ADP ratio. Nat. Commun. 4, 2550 (2013).
19. Patriarchi, T. et al. Ultrafast neuronal imaging of dopamine dynamics with designed genetically encoded sensors. Science 360 (2018).
20. Paladini, C. A. & Roeper, J. Generating bursts (and pauses) in the dopamine midbrain neurons. Neuroscience 282, 109-121 (2014).
21. Roeper, J. Dissecting the diversity of midbrain dopamine neurons. Trends Neurosci. 36, 336-342, (2013).
22. Yu, H. et al. MinK-related peptide 1: A beta subunit for the HCN ion channel subunit family enhances expression and speeds activation. Circ. Res. 88, E84-87 (2001).
23. Klaus, A., Alves da Silva, J. & Costa, R. M. What, If, and When to Move: Basal Ganglia Circuits and Self-Paced Action Initiation. Annu. Rev. Neurosci. 42, 459-483 (2019).
24. Albin, R. L., Young, A. B. & Penney, J. B. The functional anatomy of basal ganglia disorders. Trends Neurosci. 12, 366-375 (1989).
25. Wichmann, T. Changing views of the pathophysiology of Parkinsonism. Mov. Disord. 34, 1130-1143 (2019).
26. Baker, K. A. et al. Simultaneous intrastriatal and intranigral dopaminergic grafts in the parkinsonian rat model: role of the intranigral graft. J. Comp. Neurol. 426, 106-116 (2000).
27. Mukhida, K. et al. Enhancement of sensorimotor behavioral recovery in hemiparkinsonian rats with intrastriatal, intranigral, and intrasubthalamic nucleus dopaminergic transplants. J. Neurosci. 21 (2001).
28. Robertson, G. S. & Robertson, H. A. D1 and D2 dopamine agonist synergism: separate sites of action? Trends in Pharmacological Sciences 8, 295-299 (1987).
29. Coune, P. G., Schneider, B. L. & Aebischer, P. Parkinson's disease: gene therapies. Cold Spring Harb. Perspect. Med. 2, a009431 (2012).
30. Sarre, S. et al. Biotransformation of L-DOPA to dopamine in the substantia nigra of freely moving rats: effect of dopamine receptor agonists and antagonists. J. Neurochem. 70, 1730-1739 (1998).
31. Diederich, N. J. et al. Parkinson's disease: Is it a consequence of human brain evolution? Mov. Disord. 34, 453-459 (2019).
32. Pacelli, C. et al. Elevated Mitochondrial Bioenergetics and Axonal Arborization Size Are Key Contributors to the Vulnerability of Dopamine Neurons. Curr. Biol. 25, 2349-2360 (2015).
33. Berthet, A. et al. Loss of mitochondrial fission depletes axonal mitochondria in midbrain dopamine neurons. J. Neurosci. 34, 14304-14317 (2014).
34. Collier, T. J., Kanaan, N. M. & Kordower, J. H. Ageing as a primary risk factor for Parkinson's disease: evidence from studies of non-human primates. Nat. Rev. Neurosci. 12, 359-366 (2011).
35. Kordower, J. H. & Bjorklund, A. Trophic factor gene therapy for Parkinson's disease. Mov. Disord.28, 96-109 (2013).
36. Martínez-Reyes, I. & Chandel, N. S. Mitochondrial TCA cycle metabolites control physiology and disease. Nat. Commun. 11, 102 (2020).
37. Titov, D. V. et al. Complementation of mitochondrial electron transport chain by manipulation of the NAD+/NADH ratio. Science 352, 231-235 (2016).
38. Kordower, J. H. et al. Disease duration and the integrity of the nigrostriatal system in Parkinson's disease. Brain: A Journal of Neurology 136, 2419-2431 (2013).
39. Kovaleski, R. F. et al. Dysregulation of external globus pallidus-subthalamic nucleus network dynamics in parkinsonian mice during cortical slow-wave activity and activation. J. Physiology 598, 1897-1927 (2020).
40. Kravitz, A. V. et al. Regulation of parkinsonian motor behaviours by optogenetic control of basal ganglia circuitry. Nature 466, 622-626 (2010).
41. Gerfen, C. R. & Surmeier, D. J. Modulation of striatal projection systems by dopamine. Annu. Rev. Neurosci. 34, 441-466 (2011).
42. Cheramy, A., Leviel, V. & Glowinski, J. Dendritic release of dopamine in the substantia nigra. Nature 289, 537-542 (1981).
43. Crittenden, J. R. et al. Striosome-dendron bouquets highlight a unique striatonigral circuit targeting dopamine-containing neurons. Proc. Natl. Acad. Sci. USA 113, 11318-11323 (2016).
44. Rommelfanger, K. S. & Wichmann, T. Extrastriatal dopaminergic circuits of the Basal Ganglia.Front. Neuroanat. 4, 139 (2010).
45. Cáceres-Chávez, V. A. et al. Acute dopamine receptor blockade in substantia nigra pars reticulata: a possible model for drug-induced Parkinsonism. J. Neurophysiol. 120, 2922-2938 (2018).
46. de Jesús Aceves, J. et al. Dopaminergic presynaptic modulation of nigral afferents: its role in the generation of recurrent bursting in substantia nigra pars reticulata neurons. Front. Syst. Neurosci. 5, 6 (2011).
47. Smith, Y. & Kieval, J. Z. Anatomy of the dopamine system in the basal ganglia. Trends in Neurosciences 23, S28-S33 (2000).

1. A method of treating Parkinson's disease (PD) comprising increasing expression of an aromatic amino acid decarboxylase (AADC) polypeptide in the substantia nigra in a subject suffering from PD.

2. The method of claim 1, wherein AADC polypeptide is capable of catalyzing the conversion of L-DOPA to dopamine.

3. The method of claim 2, wherein the AADC polypeptide comprises 100% sequence identity with SEQ ID NO: 1.

4. The method of claim 1, wherein increasing expression of the AADC polypeptide in the substantia nigra comprises administering an agent to the subject that produces localized increased expression of AADC.

5. The method of claim 4, wherein the agent comprises a nucleic acid encoding the AADC polypeptide and administration results in expression of the AADC polypeptide from the nucleic acid.

6. The method of claim 5, wherein the nucleic acid comprises 70% sequence identity with SEQ ID NO: 2.

7. The method of claim 5, wherein the agent comprises a vector containing the nucleic acid encoding AADC.

8. The method of claim 7, wherein the vector is selected from a lipid nanoparticle, a plasmid, a transposon, an adeno-associated virus (AAV) vector, an adenovirus, a retrovirus, an integrating lentiviral vector (LVV), and a non-integrating LVV.

9. The method of claim 4, wherein the agent is administered by injection into the substantia nigra.

10. The method of claim 9, wherein injection consists of a single injection into the substantia nigra.

11. The method of claim 10, wherein another injection of the agent is not performed for at least 1 week prior to or after the single injection.

12. The method of claim 10, wherein another injection of the agent is not performed for at least 30 days prior to or after the single injection.

13. The method of one of claims 1-12, further comprising administering levodopa to the subject.

14. The method of claim 13, further comprising administering carbidopa to the subject.

15. Use of a nucleic acid or vector encoding AADC in the manufacture of a medicament for treatment of PD by administration to the substantia nigra.

16. Use of a nucleic acid or vector encoding AADC as a medicament for treatment of PD by administration to the substantia nigra.


SEQUENCE LISTING (2 sequences)

SEQ ID NO: 1 - protein, 480 aa, Homo sapiens
MNASEFRRRGKEMVDYMANYMEGIEGRQVYPDVEPGYLRPLIPAAAPQEPDTFEDIINDVEKIIMPGVTHWHSPYFFAYFPTASSYPAMLADMLCGAIGCIGFSWAASPACTELETVMMDWLGKMLELPKAFLNEKAGEGGGVIQGSASEATLVALLAARTKVIHRLQAASPELTQAAIMEKLVAYSSDQAHSSVERAGLIGGVKLKAIPSDGNFAMRASALQEALERDKAAGLIPFFMVATLGTTTCCSFDNLLEVGPICNKEDIWLHVDAAYAGSAFICPEFRHLLNGVEFADSFNFNPHKWLLVNFDCSAMWVKKRTDLTGAFRLDPTYLKHSHQDSGLITDYRHWQIPLGRRFRSLKMWFVFRMYGVKGLQAYIRKHVQLSHEFESLVRQDPRFEICVEVILGLVCFRLKGSNKVNEALLQRINSAKKIHLVPCHLRDKFVLRFAICSRTVESAHVQRAWEHIKELAADVLRAERE

SEQ ID NO: 2 - DNA, 1964 bp, Homo sapiens
  source 1..1964: mol_type = genomic DNA
aattcgggcacgagggaggacagagagcaagtcactcccggctgcctttttcacctctgacagagcccagacaccatgaacgcaagtgaattccgaaggagagggaaggagatggtggattacgtggccaactacatggaaggcattgagggacgccaggtctaccctgacgtggagcccgggtacctgcggccgctgatccctgccgctgcccctcaggagccagacacgtttgaggacatcatcaacgacgttgagaagataatcatgcctggggtgacgcactggcacagcccctacttcttcgcctacttccccactgccagctcgtacccggccatgcttgcggacatgctgtgcggggccattggctgcatcggcttctcctgggcggcaagcccagcatgcacagagctggagactgtgatgatggactggctcgggaagatgctggaactaccaaaggcatttttgaatgagaaagctggagaagggggaggagtgatccagggaagtgccagtgaagccaccctggtggccctgctggccgctcggaccaaagtgatccatcggctgcaggcagcgtccccagagctcacacaggccgctatcatggagaagctggtggcttactcatccgatcaggcacactcctcagtggaaagagctgggttaattggtggagtgaaattaaaagccatcccctcagatggcaacttcgccatgcgtgcgtctgccctgcaggaagccctggagagagacaaagcggctggcctgattcctttctttatggttgccaccctggggaccacaacatgctgctcctttgacaatctcttagaagtcggtcctatctgcaacaaggaagacatatggctgcacgttgatgcagcctacgcaggcagtgcattcatctgccctgagttccggcaccttctgaatggagtggagtttgcagattcattcaactttaatccccacaaatggctattggtgaattttgactgttctgccatgtgggtgaaaaagagaacagacttaacgggagcctttagactggaccccacttacctgaagcacagccatcaggattcagggcttatcactgactaccggcattggcagataccactgggcagaagatttcgctctttgaaaatgtggtttgtatttaggatgtatggagtcaaaggactgcaggcttatatccgcaagcatgtccagctgtcccatgagtttgagtcactggtgcgccaggatccccgctttgaaatctgtgtggaagtcattctggggcttgtctgctttcggctaaagggttccaacaaagtgaatgaagctcttctgcaaagaataaacagtgccaaaaaaatccacttggttccatgtcacctcagggacaagtttgtcctgcgctttgccatctgttctcgcacggtggaatctgcccatgtgcagcgggcctgggaacacatcaaagagctggcggccgacgtgctgcgagcagagagggagtaggagtgaagccagctgcaggaatcaaaaattgaagagagatatatctgaaaactggaataagaagcaaataaatatcatcctgccttcatggaactcagctgtctgtggcttcccatgtctttctccaaagttatccagagggttgtgattttgtctgcttagtatctcatcaacaaagaaatattatttgctaattaaaaagttaatcttcatggccatagcttttattcattagctgtgatttttgttgattaaaacattatagattttcatgttcttgcagtcatcagaagtggtaggaaagcctcactgatatattttccagggcaatcaatgttcacgcaacttgaaattatatctgtggtcttcaaattgtcttttgtcatgtggctaaatgcctaataaacaattcaagtgaaatactaaaaaaaaaaaaaaaaaaaaaa